SEQUENCE LISTING (54 sequences)

SEQ ID NO: 1 - protein, 51 aa, Artificial Sequence
  GARP peptide
DLRENKLLHFPDLAVFPRLIYLNVSNNLIQLPAGLPRGSEDLHAPSEGWSA

SEQ ID NO: 2 - protein, 11 aa, Artificial Sequence
  TAT peptide
YGRKKRRQRRR

SEQ ID NO: 3 - protein, 34 aa, Artificial Sequence
  CPP Peptide
QAATATRGRSAASRPTERPRAPARSASRPRRPVE

SEQ ID NO: 4 - protein, 16 aa, Artificial Sequence
  CPP Peptide
RQIKIWFQNRRMKWKK

SEQ ID NO: 5 - protein, 7 aa, Artificial Sequence
  CPP Peptide
RRMKWKK

SEQ ID NO: 6 - protein, 16 aa, Artificial Sequence
  CPP Peptide
RRWRRWWRRWWRRWRR

SEQ ID NO: 7 - protein, 18 aa, Artificial Sequence
  CPP Peptide
RGGRLSYSRRRFSTSTGR

SEQ ID NO: 8 - protein, 11 aa, Artificial Sequence
  CPP Peptide
YGRKKRRQRRR

SEQ ID NO: 9 - protein, 9 aa, Artificial Sequence
  CPP Peptide
RKKRRQRRR

SEQ ID NO: 10 - protein, 11 aa, Artificial Sequence
  CPP Peptide
YARAAARQARA

SEQ ID NO: 11 - protein, 8 aa, Artificial Sequence
  CPP Peptide
RRRRRRRR

SEQ ID NO: 12 - protein, 8 aa, Artificial Sequence
  CPP Peptide
KKKKKKKK

SEQ ID NO: 13 - protein, 27 aa, Artificial Sequence
  CPP Peptide
  misc_feature (25)..(25): Xaa can be any naturally occurring amino acid
GWTLNSAGYLLGKINLKALAALAKXIL

SEQ ID NO: 14 - protein, 18 aa, Artificial Sequence
  CPP Peptide
LLILLRRRIRKQANAHSK

SEQ ID NO: 15 - protein, 16 aa, Artificial Sequence
  CPP Peptide
SRRHHCRSKAKRSRHH

SEQ ID NO: 16 - protein, 11 aa, Artificial Sequence
  CPP Peptide
NRARRNRRRVR

SEQ ID NO: 17 - protein, 15 aa, Artificial Sequence
  CPP Peptide
RQLRIAGRRLRGRSR

SEQ ID NO: 18 - protein, 13 aa, Artificial Sequence
  CPP Peptide
KLIKGRTPIKFGK

SEQ ID NO: 19 - protein, 10 aa, Artificial Sequence
  CPP Peptide
RRIPNRRPRR

SEQ ID NO: 20 - protein, 18 aa, Artificial Sequence
  CPP Peptide
KLALKLALKALKAALKLA

SEQ ID NO: 21 - protein, 14 aa, Artificial Sequence
  CPP Peptide
KLAKLAKKLAKLAK

SEQ ID NO: 22 - protein, 27 aa, Artificial Sequence
  CPP Peptide
GALFLGFLGAAGSTNGAWSQPKKKRKV

SEQ ID NO: 23 - protein, 21 aa, Artificial Sequence
  CPP Peptide
KETWWETWWTEWSQPKKKRKV

SEQ ID NO: 24 - protein, 23 aa, Artificial Sequence
  CPP Peptide
GALFLGWLGAAGSTMGAKKKRKV

SEQ ID NO: 25 - protein, 23 aa, Artificial Sequence
  CPP Peptide
MGLGLHLLVLAAALQGAKSKRKV

SEQ ID NO: 26 - protein, 26 aa, Artificial Sequence
  CPP Peptide
AAVALLPAVLLALLAPAAANYKKPKL

SEQ ID NO: 27 - protein, 28 aa, Artificial Sequence
  CPP Peptide
MANLGYWLLALFVTMWTDVGLCKKRPKP

SEQ ID NO: 28 - protein, 24 aa, Artificial Sequence
  CPP Peptide
LGTYTQDFNKFHTFPQTAIGVGAP

SEQ ID NO: 29 - protein, 26 aa, Artificial Sequence
  CPP Peptide
  misc_feature (24)..(24): Xaa can be any naturally occurring amino acid
DPKGDPKGVTVTVTVTVTGKGDPXPD

SEQ ID NO: 30 - protein, 14 aa, Artificial Sequence
  CPP Peptide
PPPPPPPPPPPPPP

SEQ ID NO: 31 - protein, 18 aa, Artificial Sequence
  CPP Peptide
VRLPPPVRLPPPVRLPPP

SEQ ID NO: 32 - protein, 10 aa, Artificial Sequence
  CPP Peptide
PRPLPPPRPG

SEQ ID NO: 33 - protein, 30 aa, Artificial Sequence
  CPP Peptide
SVRRRPRPPYLPRPRPPPFFPPRLPPRIPP

SEQ ID NO: 34 - protein, 21 aa, Artificial Sequence
  CPP Peptide
TRSSRAGLQFPVGRVHRLLRK

SEQ ID NO: 35 - protein, 23 aa, Artificial Sequence
  CPP Peptide
GIGKFLHSAKKFGKAFVGEIMNS

SEQ ID NO: 36 - protein, 37 aa, Artificial Sequence
  CPP Peptide
KWKLFKKIEKVGQNIRDGIIKAGPAVAVVGQATQIAK

SEQ ID NO: 37 - protein, 28 aa, Artificial Sequence
  CPP Peptide
ALWMTLLKKVLKAAAKAALNAVLVGANA

SEQ ID NO: 38 - protein, 26 aa, Artificial Sequence
  CPP Peptide
GIGAVLKVLTTGLPALISWIKRKRQQ

SEQ ID NO: 39 - protein, 14 aa, Artificial Sequence
  CPP Peptide
INLKALAALAKKIL

SEQ ID NO: 40 - protein, 33 aa, Artificial Sequence
  CPP Peptide
GFFALIPKIISSPLPKTLLSAVGSALGGSGGQE

SEQ ID NO: 41 - protein, 15 aa, Artificial Sequence
  CPP Peptide
LAKWALKQGFAKLKS

SEQ ID NO: 42 - protein, 27 aa, Artificial Sequence
  CPP Peptide
  misc_feature (23)..(23): Xaa can be any naturally occurring amino acid
SMAQDIISTIGDLVKWIIQTVNXFTKK

SEQ ID NO: 43 - protein, 41 aa, Artificial Sequence
  CPP Peptide
LLGDFFRKSKEKIGKEFKRIVQRIKQRIKDFLANLVPRTES

SEQ ID NO: 44 - protein, 20 aa, Artificial Sequence
  CPP Peptide
LKKLLKKLLKKLLKKLLKKL

SEQ ID NO: 45 - protein, 18 aa, Artificial Sequence
  CPP Peptide
KLKLKLKLKLKLKLKLKL

SEQ ID NO: 46 - protein, 18 aa, Artificial Sequence
  CPP Peptide
PAWRKAFRWAWRMLKKAA

SEQ ID NO: 47 - protein, 663 aa, Mus musculus
MSHQILLLLAMLTLGLAISQRREQVPCRTVNKEALCHGLGLLQVPSVLSLDIQALYLSGNQLQSILVSPLGFYTALRHLDLSDNQISFLQAGVFQALPYLEHLNLAHNRLATGMALNSGGLGRLPLLVSLDLSGNSLHGNLVERLLGETPRLRTLSLAENSLTRLARHTFWGMPAVEQLDLHSNVLMDIEDGAFEALPHLTHLNLSRNSLTCISDFSLQQLQVLDLSCNSIEAFQTAPEPQAQFQLAWLDLRENKLLHFPDLAVFPRLIYLNVSNNLIQLPAGLPRGSEDLHAPSEGWSASPLSNPSRNASTHPLSQLLNLDLSYNEIELVPASFLEHLTSLRFLNLSRNCLRSFEARQVDSLPCLVLLDLSHNVLEALELGTKVLGSLQTLLLQDNALQELPPYTFASLASLQRLNLQGNQVSPCGGPAEPGPPGCVDFSGIPTLHVLNMAGNSMGMLRAGSFLHTPLTELDLSTNPGLDVATGALVGLEASLEVLELQGNGLTVLRVDLPCFLRLKRLNLAENQLSHLPAWTRAVSLEVLDLRNNSFSLLPGNAMGGLETSLRRLYLQGNPLSCCGNGWLAAQLHQGRVDVDATQDLICRFGSQEELSLSLVRPEDCEKGGLKNVNLILLLSFTLVSAIVLTTLATICFLRRQKLSQQYKA

SEQ ID NO: 48 - protein, 16 aa, Homo sapiens
DLAALPRLIYLNLSNN

SEQ ID NO: 49 - protein, 17 aa, Mus musculus
PDLAVFPRLIYLNVSNN

SEQ ID NO: 50 - protein, 17 aa, Felis catus
PDLAALPRLIYLNVSNN

SEQ ID NO: 51 - protein, 17 aa, Bos taurus
PDLSALPRLIYLNASNN

SEQ ID NO: 52 - protein, 16 aa, Pongo pygmaeus
DLAALPRLIYLNLSNN

SEQ ID NO: 53 - protein, 16 aa, Rattus norvegicus
DLAMFPRLIYLNVSNN

SEQ ID NO: 54 - protein, 662 aa, Homo sapiens
MRPQILLLLALLTLGLAAQHQDKVPCKMVDKKVSCQVLGLLQVPSVLPPDTETLDLSGNQLRSILASPLGFYTALRHLDLSTNEISFLQPGAFQALTHLEHLSLAHNRLAMATALSAGGLGPLPRVTSLDLSGNSLYSGLLERLLGEAPSLHTLSLAENSLTRLTRHTFRDMPALEQLDLHSNVLMDIEDGAFEGLPRLTHLNLSRNSLTCISDFSLQQLRVLDLSCNSIEAFQTASQPQAEFQLTWLDLRENKLLHFPDLAALPRLIYLNLSNNLIRLPTGPPQDSKGIHAPSEGWSALPLSAPSGNASGRPLSQLLNLDLSYNEIELIPDSFLEHLTSLCFLNLSRNCLRTFEARRLGSLPCLMLLDLSHNALETLELGARALGSLRTLLLQGNALRDLPPYTFANLASLQRLNLQGNRVSPCGGPDEPGPSGCVAFSGITSLRSLSLVDNEIELLRAGAFLHTPLTELDLSSNPGLEVATGALGGLEASLEVLALQGNGLMVLQVDLPCFICLKRLNLAENRLSHLPAWTQAVSLEVLDLRNNSFSLLPGSAMGGLETSLRRLYLQGNPLSCCGNGWLAAQLHQGRVDVDATQDLICRFSSQEEVSLSHVRPEDCEKGGLKNINLIIILTFILVSAILLTTLAACCCVRRQKFNQQYKA

(19) United States
(12) Patent Application Publication
(10) Pub. No.: US 2021/0087240 A1
(43) Pub. Date: Mar. 25, 2021

(54) BLOCKING GARP CLEAVAGE AND METHODS OF USE THEREOF

(71) Applicant: MUSC FOUNDATION FOR RESEARCH DEVELOPMENT, Charleston, SC (US)

(72) Inventor: Zihai LI, Mount Pleasant, SC (US)

(73) Assignee: MUSC FOUNDATION FOR RESEARCH DEVELOPMENT, Charleston, SC (US)

(21) Appl. No.: 16/630,572

(22) PCT Filed: Jul. 19, 2018
(86) PCT No.: PCT/US2018/042873

§ 371 Date: Jan. 13, 2020

Related U.S. Application Data
(60) Provisional application No. 62/534,534, filed on Jul. 19, 2017.

(51) Int. Cl. C07K 14/47; A61K 35/17; C07K 16/28; A61P 35/04

(52) U.S. Cl. CPC C07K 14/4703; A61K 35/17; A61K 2039/505; A61P 35/04; C07K 16/2818

(57) ABSTRACT

Provided herein are methods of inhibiting GARP cleavage, such as by administering a GARP peptide or a direct thrombin inhibitor. Further provided herein are methods of treating cancer by inhibiting GARP cleavage as well as methods of identifying subjects with platelet activation who would benefit from inhibition of GARP cleavage.

The present application claims the priority benefit of U.S. Provisional Application Ser. No. 62/534,534, filed Jul. 19, 2017, the entire contents of which are hereby incorporated by reference.

This invention was made with government support under Grant No. CA186866 awarded by the National Cancer Institutes. The government has certain rights in the invention.

### INCORPORATION OF SEQUENCE LISTING

The sequence listing that is contained in the file named “MESCP0107WO.txt”, which is 24 KB (as measured in Microsoft Windows) and was created on Jul. 19, 2018, is filed herewith by electronic submission and is incorporated by reference herein

### BACKGROUND

### 1. Field

The present invention relates generally to the fields of cancer biology, immunology and medicine. More particularly, it concerns blocking Glycoprotein-A Repetitions Predominant Protein (GARP) cleavage, such as for the treatment of cancer.

### 2. Description of Related Art

Depending on the type and the aggressiveness of the tumor, the incidence of pulmonary embolism and deep venus thrombosis in cancer patients are twice the frequency of the same events in non-cancer patients (Stein et al., 2006). Tumor cells indeed produce high levels of blood coagulation factors like thromboxane A2, serine proteases, matrix metalloproteinases, prostacyclin, IL-6 and nitric oxide (NO) that stimulate platelet production, aggregation, activation and degranulation (Li, 2016; Jurasz et al., 2004). Platelets, in turn, confer to cancer cells a selective advantage by forming a “cloak” of fibrin that protects the tumor by the tumoricidal attack of NK cells (Kopp et al., 2009), neutrophils (Haselmayer et al., 2007), macrophages and CTLs (Philippe et al., 1993).

Critical mediators of platelet-induced tumor growth are the α-granules released by platelets upon activation. By mass spectrometry analyses of Platelets Releasate (PR), it has been observed that one of the most abundant soluble factors secreted by platelets is TGF-β (Rachidi et al., 2017). Interestingly, it was observed that PR contained both the latent and active form of TGF-β, and that the latter was the main suppressor of T cell mediated anti-cancer immunity (Rachidi et al., 2017). However, the mechanism employed by thrombin stimulated platelets to release active TGF-β needs to be elucidated, such as for the development of cancer therapies.

### SUMMARY

In certain embodiments, the present disclosure provides methods for inhibiting cleavage of GARP, thereby inhibiting activation of TGFβ. In some embodiments, there is provided a peptide which competitively inhibits GARP cleavage (e.g., cleavage by thrombin). In further embodiments, there are provided methods of inhibiting GARP cleavage, such as for the treatment of cancer, by administering a thrombin inhibitor. In additional embodiments, there are provided methods of treating cancer by inhibiting GARP cleavage (e.g., administering a GARP peptide and/or a thrombin inhibitor) in combination with at least a second therapy, such as an immunotherapy, such as an immune checkpoint inhibitor and/or a T cell therapy.

In one embodiment, there is provided an isolated peptide comprising an amino acid sequence with at least 80%, such as 85, 86, 87, 88, 89, 90, 91, 92, 93, 94, 95, 96, 97, 98, 99, or 100%, sequence identity to SEQ ID NO:1 or SEQ ID NO:54, wherein said peptide has a length of less than 100 residues and the sequence comprises two PR cleavage sites. In some aspects, the peptide comprises an amino acid sequence with at least 95% sequence identity to SEQ ID NO:1. In some aspects, the peptide comprises the sequence of SEQ ID NO:1 or SEQ ID NO:54. In certain aspects, the peptide consists of the sequence of SEQ ID NO:1 or SEQ ID NO:54. In specific aspects, the peptide inhibits, such as competitively inhibits, binding of GARP to thrombin.

In some aspects, the peptide comprises less than 90, 80, 70, 60, 50, 40, 30, or 20 residues. In certain aspects, the peptide comprises at least 10, 15, 20, 25, 30, 35, 40, 45 or 50 residues. The peptide may have a length of about 10, 11, 12, 13, 14, 15, 20, 25, 30, 35, 40, 45, 50, or 51 residues. In some aspects, the peptide may be a fragment of SEQ ID NO:1 or SEQ ID NO:54, such as a fragment comprising one or more thrombin cleavage sites. The fragment may comprise 10-15, 15-20, 20-25, or 25-30 residues. In some aspects, the peptide is fused to a cell-penetrating peptide. In particular aspects, the peptide comprises at least one thrombin binding site, such as two or three thrombin binding sites.

Further provided herein are isolated nucleic acids encoding the peptides of the embodiments. Also provided herein is a vector comprising a contiguous sequence comprising said nucleic acid.

In another embodiment, there is provided a pharmaceutical composition comprising (a) a peptide that acts as a competitive inhibitor of GARP cleavage and (b) a pharmaceutically acceptable carrier, buffer or diluent. In some aspects, the peptide is a peptide of the embodiments (e.g., a GARP peptide which blocks cleavage of GARP).

Also provided herein is a host cell comprising one or more peptides of the embodiments (e.g., a peptide with at least 80%, such as 85, 86, 87, 88, 89, 90, 91, 92, 93, 94, 95, 96, 97, 98, 99, or 100%, sequence identity to SEQ ID NO:1 or SEQ ID NO:54 or a fragment thereof). In some aspects, the host cell is a mammalian cell, a yeast cell, a bacterial cell, a ciliate cell or an insect cell.

In another embodiment, there is provide a method for treating cancer comprising administering an effective amount of a peptide of the embodiments (e.g., a GARP peptide which blocks binding of thrombin to GARP, particularly a peptide with at least 80%, such as 85, 86, 87, 88, 89, 90, 91, 92, 93, 94, 95, 96, 97, 98, 99, or 100%, sequence identity to SEQ ID NO:1 or SEQ ID NO:54 or a fragment thereof) to the subject. In some aspects, administering the peptide results in a decrease in the cleavage of GARP.

In certain aspects, the cancer is a breast cancer, lung cancer, head & neck cancer, prostate cancer, esophageal cancer, tracheal cancer, skin cancer brain cancer, liver cancer, bladder cancer, stomach cancer, pancreatic cancer, ovarian cancer, uterine cancer, cervical cancer, testicular cancer, colon cancer, rectal cancer, skin cancer or a hematological cancer. In particular aspects, the cancer is colon cancer, melanoma, breast cancer, or prostate cancer. In specific aspects, the cancer is metastatic. In specific aspects, the cancer is a GARP positive cancer.

In some aspects, the peptide is administered systemically. In certain aspects, the peptide is administered intravenously, intradermally, intratumorally, intramuscularly, intraperitoneally, subcutaneously, or locally.

In additional aspects, the method further comprises administering at least a second anticancer therapy to the subject. In some aspects, the second anticancer therapy is a surgical therapy, chemotherapy, radiation therapy, cryotherapy, hormonal therapy, immunotherapy or cytokine therapy. In certain aspects, the second anticancer therapy comprises an immunotherapy, such as an immune checkpoint inhibitor and/or a T cell therapy. In some aspects, the immune checkpoint inhibitor is an anti-PD1 antibody.

In another embodiment, there is provided a composition comprising an effective amount of a peptide according to the embodiments (e.g., a GARP peptide which blocks binding of thrombin to GARP, particularly a peptide with at least 80%, such as 85, 86, 87, 88, 89, 90, 91, 92, 93, 94, 95, 96, 97, 98, 99, or 100%, sequence identity to SEQ ID NO:1 or SEQ ID NO:54 or a fragment thereof) for use in the treatment of a cancer.

A further embodiment provides a method for treating cancer in a subject comprising administering an effective amount of a direct thrombin inhibitor to the subject. In some aspects, administering the direct thrombin inhibitor decreases cleavage of GARP. In certain aspects, the cancer is a GARP positive cancer. In specific aspects, the subject is identified to have a high level of soluble GARP.

In some aspects, the direct thrombin inhibitor is hirudin, bivalirudin, lepirudin, desirudin, argatroban, dabigatran, dabigatran etexilate, melagatran, or ximelagatran. In particular aspects, the direct thrombin inhibitor is dabigatran etexilate.

In certain aspects, the cancer is colon cancer, melanoma, breast cancer, or prostate cancer. In particular aspects, the cancer is metastatic.

In additional aspects, the method further comprises administering at least a second anticancer therapy to the subject. In some aspects, the second anticancer therapy is a surgical therapy, chemotherapy, radiation therapy, cryotherapy, hormonal therapy, immunotherapy or cytokine therapy. In certain aspects, the second anticancer therapy comprises an immunotherapy. In certain aspects, the immunotherapy is T cell therapy. In other aspects, the immunotherapy comprises an immune checkpoint inhibitor. In some aspects, the immunotherapy comprises T cell therapy and an immune checkpoint inhibitor. In some aspects, the immune checkpoint inhibitor is an anti-PD1 antibody and/or an anti-CTLA4 antibody. In certain aspects, the anti-PD1 antibody is nivolumab, pembrolizumab, CT-011, BMS 936559, MPDL328OA or AMP-224.

In certain aspects, the direct thrombin inhibitor is administered intravenously, intradermally, intratumorally, intramuscularly, intraperitoneally, subcutaneously, or locally.

In another embodiment, there is provided a composition comprising an effective amount of a direct thrombin inhibitor for use in the treatment of a cancer.

In yet another embodiment, there is provide a method of treating cancer in a subject comprising administering an effective amount of a peptide according to the embodiments (e.g., a GARP peptide which blocks binding of thrombin to GARP, particularly a peptide with at least 80% sequence identity to SEQ ID NO:1 or a fragment thereof) and/or a direct thrombin inhibitor to the subject, wherein the subject is identified to have a high level of soluble GARP. In some aspects, the subject is further identified to have a high concentration of TGFβ. In certain aspects, the direct thrombin inhibitor is hirudin, bivalirudin, lepirudin, desirudin, argatroban, dabigatran, dabigatran etexilate, melagatran, or ximelagatran. In particular aspects, the direct thrombin inhibitor is dabigatran etexilate. In some aspects, the cancer is colon cancer, melanoma, breast cancer, or prostate cancer. In some aspects, the cancer is metastatic.

In additional aspects, the method further comprises administering at least a second anticancer therapy to the subject. In some aspects, the second anticancer therapy is a surgical therapy, chemotherapy, radiation therapy, cryotherapy, hormonal therapy, immunotherapy or cytokine therapy. In certain aspects, the second anticancer therapy comprises an immunotherapy. In some aspects, the immunotherapy is T cell therapy. In certain aspects, the immunotherapy comprises an immune checkpoint inhibitor. In some aspects, the immunotherapy comprises a T cell therapy and an immune checkpoint inhibitor. In certain aspects, the immune checkpoint inhibitor is an anti-PD1 antibody and/or an anti-CTLA4 antibody. In some aspects, the anti-PD1 antibody is nivolumab, pembrolizumab, CT-011, BMS 936559, MPDL328OA or AMP-224.

In some aspects, the direct thrombin inhibitor is administered intravenously, intradermally, intratumorally, intramuscularly, intraperitoneally, subcutaneously, or locally. In certain aspects, the direct thrombin inhibitor is administered simultaneously with the immunotherapy. In other aspects, the direct thrombin inhibitor is administered prior to or after the immunotherapy. As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1E: GARP deletion on platelets does not alter platelets activation and number. (A) Polymerase chain reaction (PCR) showing the excision of exon 1 from GARP gene that results in a smaller DNA fragment. (B) Flow cytometry analysis showing the complete lack of expression of GARP in CD41+ platelets. (C) Little incision was performed on mice tail and bleeding time was evaluated. (D) Periperal blood platelet count performed by scil Vet ABC instrument. (E) Flow cytometry analysis to evaluate the p-Selectin expression of WT and GARP KO platelets upon thrombin activation. Two-tailed, independent Student's t-test was used in panels C and D.

FIGS. 2A-2F: Platelet-derived GARP/TGF-β complex blunts adoptive T cell therapy of melanoma. (A) Tumor growth of WT and Pf4creGARPf/f mice (n=7-8) subcutaneously injected with B16-F1. (B) Experiment design: on day 0. Splenocytes from Thy1.1+ Pmel transgenic mice were culture for 3 days and injected on day 10 in congenic Thy1.2 mice previously conditioned with Cy on day 9. (C) Tumor growth curves of mice. (D) Kaplan-Mayer survival curve in B16-F1 adoptively transferred bearing mice. (E) The frequency of Pmel cells in mice was enumerated 3 weeks post-adoptive transfer of T cells by flow cytometry in the peripheral blood (CD8+Thy1.1+/total CD8+). (F) IFN-γ producing ability of antigen-specific donor T cells (Pmel) from indicated mice 3 weeks after T cell transfer. Repeated measures ANOVA was used in panel A and C. Two-tailed, independent Student's t-test was used in panels E and F.

FIGS. 3A-3B: Platelet-intrinsic GARP plays critical roles in generating active TGF-β. Serum levels of active (A) and total (B) TGF-β were measured by ELISA in B16-F1 bearing mice (n=6-8 per group). Comparison was performed using two-tailed, independent Student's t-test.

FIGS. 4A-4F: Targeting platelet-derived GARP/TGF-β complex improves MC38 tumor control. (A) WT or Platelet GARP KO mice (n=5 each group) were injected in the right flank with 1×106 MC38 colon cancer cells. Tumor size was measured every 3 days with digital vernier caliper. (B) Kaplan-Mayer survival curve in MC38-bearing mice. (C) In a separate experiment, 6 weeks after MC38 injection, mice were sacrificed and the primary tumors were resected and weighed. (D) The inset shows the photographs of primary tumors resected from mice 6 weeks after injection. Serum was obtained from mice 6 weeks after MC38 injection and (E) active and total (F) TGF-β was measured by ELISA. Repeated measures two-way ANOVA was used in panel A; Kaplan-Meier curves and log rank tests were used in panel B. Two-tailed, independent Student's t-test was used in panels C, E, and F.

FIGS. 5A-5C: Targeting platelet-derived GARP/TGF-β complex results in reduction of TGF-β activity in the tumor microenvironment. (A) IHC for p-SMAD-2/3 in MC38 tumors from indicated mice; representative images are shown. Scale bar: 12.5 μm (B) Independent histopathological quantification of p-SMAD-2/3 staining intensity from panel (E) (n=4 per group). (C) Percentage of regulatory T cells CD25+ FOXP3+ in the CD4+ tumor-infiltrating lymphocytes (TIL) from the indicated mice. Two-tailed, independent Student's t-test was used in panels B and C.

FIGS. 6A-6F: Platelet GARP is increased upon thrombin stimulation and enhances active TGF-β release in Platelets Releasate. (A) Flow cytometry analysis of surface GARP/LAP complex on WT and GARP KO platelets with and without thrombin stimulation. (B) Schematic representation of the passages required for PR isolation. (C) PR from WT and GARP KO platelets was obtained with and without thrombin stimulation. PR was analyzed by western Blot in non-reducing and non-denaturating conditions. (D) PR from WT and GARP KO platelets was obtained with and without thrombin stimulation. TGF-β was quantified by ELISA. (E) PR from WT animals was stimulated with increasing concentration of Thrombin and analyzed by WB in reducing and denaturating conditions. (F) PR from WT and GARP KO platelets was obtained with and without thrombin stimulation. Soluble GARP was quantified by ELISA. Two-tailed, independent Student's t-test was used in panels D and F.

FIGS. 7A-7G: Direct thrombin inhibitor Dabigatran Etexilate reduces platelet GARP expression and protects against melanoma and colon cancer. (A) Flow cytometry analysis of surface GARP/LAP complex on murine WT platelets stimulated with and without thrombin and Dabigratan Etexilate. (B) Tumor growth curves of mice subcutaneously injected with B16-F1 melanoma cells and daily treated with 3 mg/mouse of Dabigratan Etexilate. (C) Tumor growth curves of mice subcutaneously injected with MC38 colon cancer cells and daily treated with 3 mg/mouse of Dabigratan Etexilate. (D) Serum was obtained from tumor bearing MC38 colon cancer and active (D) and total TGF-β (E) was measured by ELISA. (F) Serum was obtained from tumor bearing MC38 colon cancer and soluble GARP was measured by ELISA. (G) Percentage of regulatory T cells CD25+ FOXP3+ in the CD4+ tumor-infiltrating CD4+ lymphocytes (TIL) from the indicated mice. Two-tailed, independent Student's t-test was used in panel D, E, F, and G. Repeated measures two-way ANOVA was used in panel A Direct thrombin inhibitor Dabigatran Etexilate in combination with anti-PD1 blockade antibody reduces tumor burden in MC38 tumor model.

FIGS. 8A-8E: Direct thrombin inhibitor Dabigatran Etexilate in combination with anti-PD1 blockade antibody reduces tumor burden in MC38 tumor model. (A) Experimental design for Dabigatran and PD1 blocking antibody combination therapy. (B) Tumor growth curves of mice subcutaneously injected with MC38 colon cancer cells and treated with Dabigatran alone, with PD1 blocking antibody, with the combination of Dabigatran and PD1 blocking antibody or left untreated. (C) Survival curve in MC38-bearing mice treated with Dabigatran alone, with PD1 blocking antibody, with the combination of Dabigatran and PD1 blocking antibody or left untreated. Repeated measurement 2 way Anova was performed in B. Log-rank (Mantel-Cox) was performed in C. (D) Serum was collected from the mice under the dual treatment and analyzed for TGFβ concentration by TGFβ ELISA. (E) Platelets count of the mice under dual treatment. Repeated measurement 2-way ANOVA was performed in B. Log-rank (Mantel-Cox) was performed in C. Two-tailed, independent Student's t-test was used in panel D and E.

FIGS. 9A-9C: GARP is cleaved on the cell surface. (A) Western Blot analysis of GARP expressing PreB cells WT or gp96 KD. Cell lysates were analyzed by using antibodies against mouse GARP and mouse gp96 (B) Parallel western Blot analysis of cells lysates and conditioned media of PreB cells expressing GARP or control vector. (C) Mass Spectrometry analysis of the fragment present in the conditioned medium indicated in the box.

FIGS. 10A-10D: Surface GARP is cleaved by thrombin. (A) GARP amino acidic sequence (SEQ ID NO:47): the part in bold indicates the sequence found in the conditioned medium; the arrows indicate the predicted thrombin cutting sites based on ExPASy analysis. (B) GARP expressing PreB cells were digested with increasing concentration of thrombin (0, 1, 2, and 4 μg in 25 μl), followed by western blot analysis. (C) Sh-RNA mediated KD of thrombin in PreB cells expressing GARP followed by western blot analysis. (D) Western blot analysis of WT and GARP KO platelets treated with thrombin.

FIGS. 11A-11B: GARP upregulates thrombin gene expression. (A) RT-PCR analysis of Lrrc32 and thrombin gene expression in PreB cells expressing GARP or control vector. (B) Regression analysis of between GARP and thrombin expression. Data obtained from TCGA, Breast Cancer proteomic database.

FIGS. 12A-12E: Thrombin cleaves GARP at the amino acid position 267 and 286 between proline and arginine. (A) Surface Flow cytometry analysis for GARP of WT PreB cells and GARP expression in PreB cells expressing WT GARP, GARP with single point mutation 267 aa, GARP with single point mutation 286 aa, GARP with double point mutation 267/286 aa (DM), and relative control vector. (B) Western Blot analysis of cells lysates and conditioned media of PreB cells expressing WT GARP, GARP with single point mutation 267 aa, GARP with single point mutation 286 aa, or GARP with double point mutation 267/286 aa using antibody against GARP. (C) PreB cells expressing WT or DM GARP were digested with increasing concentration of thrombin (0, 1, 2, and 4 μg in 25 μl), followed by western blot analysis. (D) Recombinant fragment of GARP containing the two Proline Arginine thrombin binding sites (T250; SEQ ID NO:1). (E) Western blotting analysis of GARP expressing PreB cells digested with 4 μg of thrombin in presence and absence of 4 μg of T250. Thrombin-mediated cleavage facilitates latent TGF-β release from cell surface, however does not affect mature TGF-β formation.

FIGS. 13A-13C: Thrombin-mediated cleavage facilitates latent TGF-β release from cell surface, however does not affect mature TGF-β formation. (A) Surface Flow cytometry analysis for LAP of WT PreB cells and GARP expression in PreB cells expressing WT GARP, GARP with single point mutation 267 aa, GARP with single point mutation 286 aa, GARP with double point mutation 267/286 aa (DM), and relative control vector. (B) Parallel western Blot analysis of cells lysates and conditioned media of PreB cells expressing WT GARP, GARP with single point mutation 267 aa, GARP with single point mutation 286 aa, or GARP with double point mutation 267/286 aa using antibody against TGF-β. (C) Total TGF-β ELISA of conditioned medium of PreB cells expressing WT GARP, GARP with single point mutation 267 aa, GARP with single point mutation 286 aa, or GARP with double point mutation 267/286. Statistical significance was analyzed by two-tailed T-test in C.

FIGS. 14A-14E: Recombinant GARP protein is bound to latent TGF-β and is cleaved by thrombin. (A) Western blot analysis of sGARP in reducing denaturating (D) and non-reducing non-denaturating (ND) conditions. Antibody versus GARP and TGF-β were used. (B) Total TGF-β ELISA of 3 serial dilutions of soluble GARP-Fc. (C-E) Western blot analysis of lug soluble GARP digested with increasing concentrations of thrombin (0, 1, 2, and 4 μg in 25 μl) in reducing and non-reducing conditions. Antibody versus GARP and TGF-β were used.

FIGS. 15A-15J: Soluble GARP enhances TGF-β through αV integrins (A) Flow cytometry analysis of GFP p-SMAD NMuMG cells stimulated with soluble GARP. (B) Flow cytometry analysis of surface αV integrins in NMuMG cells. (C) RT-PCR analysis of aV and 13 integrins expressed on NMuMG cells. (D) Flow cytometry analysis of GFP expression in NMuMG cells stimulated with 8 μg of soluble GARP in presence of increasing concentration of RGD peptide. (E) Flow cytometry analysis of NMuMG GFP cells reporter cells for p-SMAD2/3. Cells were forced to express either WT GARP, or double mutant GARP and GFP expression was analyzed by flow cytometry. (F) Molecular model of GARP and thrombin interaction and resulting N+ terminal soluble GARP: the dimeric structure of latent TGFβ and GARP are indicated. (G) Western blot analysis of recombinant N+ terminal GARP/LTGFβ complex. Membrane was blot for anti-GARP and anti-TGFβ. (H) Molecular modeling of the interaction between N+ terminal GARP/Latent TGFbeta and integrins. (I) Dose dependent stimulation of NMuMG GFP p-SMAD 2/3 reporter cells with recombinant N+ terminal GARP/LTGFβ; (J) Stimulation of NMuMG GFP p-smad2/3 reporter cells with recombinant N+ terminal GARP/LTGFβ in presence of RGD or RGE peptide.

FIGS. 16A-16B: Soluble GARP/latent TGF-β complex is internalized by cells through integrins. (A) Upper panel: surface flow cytometry analysis and confocal pictures of NMuMG cells stimulated with 1 μg of sGARP; Lower panel: intracellular flow cytometry analysis and confocal pictures of NMuMG cells stimulated with 1 μg of sGARP. (B) Confocal pictures of fixed and permeabilized NMuMG cells stimulated with 1 μg of sGARP in presence or absence of RGD peptide.

FIGS. 17A-17C: GARP/latent TGF-β complex is released in exosomes. (A) Western blot analysis in reducing and denaturating conditions of exosomes isolated from conditioned medium of 293 HEK TGF-β with and without GARP expression. Anti-GARP antibody was used. (B) Western blot analysis in non-reducing and non-denaturating condition of exosomes isolated from conditioned medium of 293 HEK TGF-β with and without GARP expression. Anti-GARP and anti-TGF-β antibodies were used. (C) Western blot analysis of CD63 in reducing and denaturating conditions of exosomes isolated from conditioned medium of 293 HEK TGF-β with and without GARP expression.

FIGS. 18A-18E: GARP cleavage releases platelet active TGFβ. (A) WT and GARP KO platelets were stimulated for 1 hour with 1 U/ml of thrombin followed by Western blot analysis of platelet lysate and releasate. (B) TGFβ ELISA performed on releasate of WT and GARP KO platelet treated 1 hour with 1 U/ml of thrombin. (C) Active and total TGFβ ELISA performed on serum collected from WT and platelet GARP KO mice daily treated with 3 mg/mouse of Dabigatran Etexilate. (D) TGFβ ELISA of mouse platelets stimulated with thrombin in presence of either Dabigatran or T250 blocking peptide. (E) Flow cytometry analysis of CD62p (p-selectin) expression in mouse platelets stimulated for 1 hour in PBS in presence of either 1 U/ml of mouse thrombin, 1 μg/ml of T250 peptide, or both. Two-tailed, independent Student's t-test was used in panel B, C, D, and E.

FIGS. 19A-19E: Thrombin cleaves human GARP enhancing active TGFβ releases from platelets (A) Alignment of one conserved thrombin cutting site on GARP protein among difference species (SEQ ID NOs:48-53). (B) Human GARP sequence (SEQ ID NO:54) with the predicted thrombin binding sites (PR). (C) Digestion of human recombinant thrombin with dose dependent human alpha thrombin. (D) Western Blot analysis of releasate from human platelets stimulated either by shaking, or with alpha thrombin, or with ADP. (E) TGFβ analysis of releasate of human platelets stimulated with thrombin in presence of Dabigatran. Two-tailed, independent Student's t-test was used in panel E.

FIGS. 20A-20C: Direct thrombin inhibitor Dabigatran etexilate decreases systemic active TGFβ and has anti-cancer effect in MC38 colorectal cancer. (A) Tumor growth curves of mice subcutaneously injected with MC38 colon cancer cells and daily treated with 3 mg/mouse of Dabigatran etexilate. (B) Tumor growth curves of mice subcutaneously injected with B16-F1 melanoma cells and daily treated with 3 mg/mouse of Dabigatran etexilate. (C) TGFβ ELISA on serum of WT B6 mice treated for one week with daily gavage of 3 mg/mouse of Dabigatran etexilate. Repeated measurement 2-way ANOVA was performed in A and B.

### DESCRIPTION OF ILLUSTRATIVE EMBODIMENTS

GARP (also referred to as Leucine-rich repeat protein 32 (LRRC32)) is a latent-TGF-β receptor expressed abundantly on Tregs, platelets, and cancer cells. It has been demonstrated that GARP can be shed form the cell surface and released extracellularly. It has been shown that anti-GARP proteins may be used for the treatment and diagnosis of cancer alone or in combination with immunotherapy, such as T cell therapy (PCT/US2017/025037; incorporated herein by reference in its entirety).

There are several mechanisms by which proteins may be cleaved. For example, thrombin is a trypsin-like serine protease involved in the conversion of fibrinogen to fibrin. Another mechanism that cells utilize to secrete proteins is through the exosome machinery. These nanoscale vesicles of endocytic origins are secreted by most cells in the extracellular milieu where they travel until they fuse to the membrane of other cells. Exosomes mediate the intercellular communication carrying RNA, proteins, and DNA that can affect target cells.

The present studies focused on the potential surface shedding of membrane bound GARP and investigated the role of thrombin as the enzyme involved in the cleavage, such as by site specific mutagenesis. The studies herein showed that soluble GARP is formed through thrombin mediated shedding as well as exosome mediated GARP shedding.

Specifically, a novel mouse model of platelet specific knock-out of the gene encoding for GARP was employed. Additionally, the clinical potential of a target pharmacological therapy to reduce platelet surface GARP was investigated. Interestingly, it was found that GARP is proteolytically cleaved by thrombin from the cell surface to promote the release of TGFβ and, thus, thrombin can at the same time cleave GARP and activate TGFβ. In addition, the thrombin-cleavage sites of GARP were successfully mapped.

Accordingly, certain embodiments of the present disclosure provide methods for blocking GARP cleavage, such as by using a platelet inhibitor, such as a direct thrombin inhibitor, and/or a GARP peptide provided herein (e.g., T250 peptide with the sequence DLRENKLLHFPDLAVFPRLIYLNVSNNLIQLPAGLPRGSEDLHAPSEGWSA (SEQ ID NO:1). Blocking GARP cleavage can impair the release of soluble GARP from platelets and block the release of active TGFβ and, thus, block tumor growth. The inhibition of GARP cleavage can enhance the efficacy of immunotherapy, such as T cell therapy and/or immune checkpoint inhibitors. Thus, the inhibitors of GARP cleavage may be used alone or in combination with other therapies, such as immunotherapy, particularly immune checkpoint inhibitors (e.g., anti-PD1 antibody) for the treatment of cancer. In particular aspects, the thrombin inhibitor Dabigatran Etexilate, also known as Pradax, may be used which relies on the competitive and reversible binding to thrombin active site, thus impeding coagulation factor-mediated thrombin activation.

Thus, in some embodiments, the present disclosure provides methods of treating cancer through the inhibition of GARP cleavage, and consequently inhibition of TGFβ. Also provided herein are methods of determining if a subject should be administered an anti-platelet agent or inhibitor of GARP cleavage by measuring the level of soluble GARP and/or latent TGFβ.

### I. DEFINITIONS

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of an antibody that inhibits the GARP signaling. In another example, a treatment may include administration of a T cell therapy and a pharmaceutically effective amount of an anti-platelet agent (e.g., an antibody that inhibits the GARP signaling).

“Subject” and “patient” refer to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

The term “immune checkpoint” refers to a molecule such as a protein in the immune system which provides inhibitory signals to its components in order to balance immune reactions. Known immune checkpoint proteins comprise CTLA-4, PD1 and its ligands PD-L1 and PD-L2 and in addition LAG-3, BTLA, B7H3, B7H4, TIM3, MR. The pathways involving LAG3, BTLA, B7H3, B7H4, TIM3, and MR are recognized in the art to constitute immune checkpoint pathways similar to the CTLA-4 and PD-1 dependent pathways (see e.g. Pardoll, 2012; Mellman et al., 2011).

An “immune checkpoint inhibitor” refers to any compound inhibiting the function of an immune checkpoint protein. Inhibition includes reduction of function and full blockade. In particular the immune checkpoint protein is a human immune checkpoint protein. Thus the immune checkpoint protein inhibitor in particular is an inhibitor of a human immune checkpoint protein.

A polynucleotide or polynucleotide region (or a polypeptide or polypeptide region) has a certain percentage (for example, 80%, 85%, 90%, or 95%) of “sequence identity” or “homology” to another sequence means that, when aligned, that percentage of bases (or amino acids) are the same in comparing the two sequences. This alignment and the percent homology or sequence identity can be determined using software programs known in the art, for example those described in CURRENT PROTOCOLS IN MOLECULAR BIOLOGY (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60, expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR.

By “expression construct” or “expression cassette” is meant a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as a gene delivery system or gene transfer “vehicle”) refers to a macromolecule or complex of molecules comprising a polynucleotide to be delivered to a host cell, either in vitro or in vivo.

A “plasmid,” a common type of a vector, is an extra-chromosomal DNA molecule separate from the chromosomal DNA that is capable of replicating independently of the chromosomal DNA. In certain cases, it is circular and double-stranded.

An “origin of replication” (“ori”) or “replication origin” is a DNA sequence, e.g., in a lymphotrophic herpes virus, that when present in a plasmid in a cell is capable of maintaining linked sequences in the plasmid and/or a site at or near where DNA synthesis initiates. As an example, an ori for EBV includes FR sequences (20 imperfect copies of a 30 bp repeat), and preferably DS sequences; however, other sites in EBV bind EBNA-1, e.g., Rep* sequences can substitute for DS as an origin of replication (Kirshmaier and Sugden, 1998). Thus, a replication origin of EBV includes FR, DS or Rep* sequences or any functionally equivalent sequences through nucleic acid modifications or synthetic combination derived therefrom. For example, the present invention may also use genetically engineered replication origin of EBV, such as by insertion or mutation of individual elements, as specifically described in Lindner, et. al., 2008.

A “gene,” “polynucleotide,” “coding region,” “sequence,” “segment,” “fragment,” or “transgene” that “encodes” a particular protein, is a nucleic acid molecule that is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “control elements” refers collectively to promoter regions, polyadenylation signals, transcription termination sequences, upstream regulatory domains, origins of replication, internal ribosome entry sites (IRES), enhancers, splice junctions, and the like, which collectively provide for the replication, transcription, post-transcriptional processing, and translation of a coding sequence in a recipient cell. Not all of these control elements need be present so long as the selected coding sequence is capable of being replicated, transcribed, and translated in an appropriate host cell.

The term “promoter” is used herein in its ordinary sense to refer to a nucleotide region comprising a DNA regulatory sequence, wherein the regulatory sequence is derived from a gene that is capable of binding RNA polymerase and initiating transcription of a downstream (3′ direction) coding sequence. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

By “enhancer” is meant a nucleic acid sequence that, when positioned proximate to a promoter, confers increased transcription activity relative to the transcription activity resulting from the promoter in the absence of the enhancer domain.

By “operably linked” or co-expressed” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” or “co-expressed” with reference to peptide and/or polypeptide molecules means that two or more peptide and/or polypeptide molecules are connected in such a way as to yield a single polypeptide chain, i.e., a fusion polypeptide, having at least one property of each peptide and/or polypeptide component of the fusion. The fusion polypeptide is preferably chimeric, i.e., composed of heterologous molecules.

### II. INHIBITION OF GARP CLEAVAGE

Certain embodiments of the present disclosure provide methods and compositions for the inhibition of GARP cleavage, and consequently the inhibition of latent-TGFβ, such as for the treatment of cancer. The GARP cleavage may be inhibited by the administration of a peptide which blocks binding of an enzyme, such as thrombin, to GARP, particularly soluble GARP. The cleavage of GARP may be inhibited by a direct thrombin inhibitor, such as dabigatran etexilate. In some aspects, GARP cleavage may be inhibited by the administration of both a peptide provided herein and a direct thrombin inhibitor.

A. GARP Peptides

Accordingly, in some embodiments, there are provided peptides which block binding of GARP to thrombin as well as methods of their use, referred to herein as GARP peptides. These include peptides or expression vectors encoding the peptides disclosed herein as well as that structurally similar compounds (i.e., small molecules) that may be formulated to mimic the key portions of peptide.

In particular embodiments, the peptide has at least 80, 85, 90, 91, 92, 93, 94, 95, 96, 97, 98, 99, or 100% sequence identity with the sequence DLRENKLLHFPDLAVFP̅R̅LIYLNVSNNLIQLPAGLP̅R̅GSEDLHAPSEGWSA (SEQ ID NO:1). In some embodiments, the peptide has at least 80, 85, 90, 91, 92, 93, 94, 95, 96, 97, 98, 99, or 100% sequence identity with the sequence of human GARP: MRPQILLLLALLTLGLAAQHQDKVPCKMVDKKVSCQVLGLLQVPSVLPPDTETLDLS GNQLRSILASPLGFYTALRHLDLSTNEISFLQPGAFQALTHLEHLSLAHNRLAMATAL SAGGLGPLPRVTSLDLSGNSLYSGLLERLLGEAPSLHTLSLAENSLTRLTRHTFRDMP ALEQLDLHSNVLMDIEDGAFEGLP̅R̅LTHLNLSRNSLTCISDFSLQQLRVLDLSCNSIEA FQTASQPQAEFQLTWLDLRENKLLHFPDLAALP̅R̅LIYLNLSNNLIRLPTGPPQDSKGIH APSEGWSALPLSAPSGNASGRPLSQLLNLDLSYNEIELIPDSFLEHLTSLCFLNLSRNCL RTFEARRLGSLPCLMLLDLSHNALETLELGARALGSLRTLLLQGNALRDLPPYTFAN LASLQRLNLQGNRVSPCGGPDEPGPSGCVAFSGITSLRSLSLVDNEIELLRAGAFLHTP LTELDLSSNPGLEVATGALGGLEASLEVLALQGNGLMVLQVDLPCFICLKRLNLAEN RLSHLPAWTQAVSLEVLDLRNNSFSLLPGSAMGGLETSLRRLYLQGNPLSCCGNGW LAAQLHQGRVDVDATQDLICRFSSQEEVSLSHVRPEDCEKGGLKNINLIIILTFILVSAI LLTTLAACCCVRRQKFNQQYKA (SEQ ID NO:54). The PR cleavage sites are underlined.

In general, the peptides will be 60 residues or less. The overall length may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 residues. Ranges of peptide length of 10-50 residues, 15-50 residues, 20-25 residues 21-25, residues, 20-30 residues, 30-40 residues, and 35-45 residues, and 25-35 residues are contemplated. The present disclosure may utilize L-configuration amino acids, D-configuration amino acids, or a mixture thereof. While L-amino acids represent the vast majority of amino acids found in proteins, D-amino acids are found in some proteins produced by exotic sea-dwelling organisms, such as cone snails. They are also abundant components of the peptidoglycan cell walls of bacteria. D-serine may act as a neurotransmitter in the brain. The L and D convention for amino acid configuration refers not to the optical activity of the amino acid itself, but rather to the optical activity of the isomer of glyceraldehyde from which that amino acid can theoretically be synthesized (D-glyceraldehyde is dextrorotary; L-glyceraldehyde is levorotary).

One form of an “all-D” peptide is a retro-inverso peptide. Retro-inverso modification of naturally occurring polypeptides involves the synthetic assemblage of amino acids with α-carbon stereochemistry opposite to that of the corresponding L-amino acids, i.e., D-amino acids in reverse order with respect to the native peptide sequence. A retro-inverso analogue thus has reversed termini and reversed direction of peptide bonds (NH—CO rather than CO—NH) while approximately maintaining the topology of the side chains as in the native peptide sequence. See U.S. Pat. No. 6,261,569, incorporated herein by reference.

The present disclosure contemplates fusing or conjugating a cell-penetrating domain (also called a cell delivery domain, or cell transduction domain). Such domains are well known in the art and are generally characterized as short amphipathic or cationic peptides and peptide derivatives, often containing multiple lysine and arginine resides (Fischer, 2007). Of particular interest are the TAT sequence from HIV1 (YGRKKRRQRRR; SEQ ID NO: 2), and poly-D-Arg and poly-D-Lys sequences (e.g., dextrorotary residues, eight residues in length). Other cell delivery domains are shown in the table below.

TABLE 1

| | SEQ |
|---|---|
| CPP/CTD PEPTIDES | ID NO |
| QAATATRGRSAASRPTERPRAPARSASRPRRPVE | 3 |
| RQIKIWFQNRRMKWKK | 4 |
| RRMKWKK | 5 |
| RRWRRWWRRWWRRWRR | 6 |
| RGGRLSYSRRRFSTSTGR | 7 |
| YGRKKRRQRRR | 8 |
| RKKRRQRRR | 9 |
| YARAAARQARA | 10 |
| RRRRRRRR | 11 |
| KKKKKKKK | 12 |
| GWTLNSAGYLLGKINLKALAALAKXIL | 13 |
| LLILLRRRIRKQANAHSK | 14 |
| SRRHHCRSKAKRSRHH | 15 |
| NRARRNRRRVR | 16 |
| RQLRIAGRRLRGRSR | 17 |
| KLIKGRTPIKFGK | 18 |
| RRIPNRRPRR | 19 |
| KLALKLALKALKAALKLA | 20 |
| KLAKLAKKLAKLAK | 21 |
| GALFLGFLGAAGSTNGAWSQPKKKRKV | 22 |
| KETWWETWWTEWSQPKKKRKV | 23 |
| GALFLGWLGAAGSTMGAKKKRKV | 24 |
| MGLGLHLLVLAAALQGAKSKRKV | 25 |
| AAVALLPAVLLALLAPAAANYKKPKL | 26 |
| MANLGYWLLALFVTMWTDVGLCKKRPKP | 27 |
| LGTYTQDFNKFHTFPQTAIGVGAP | 28 |
| DPKGDPKGVTVTVTVTVTGKGDPXPD | 29 |
| PPPPPPPPPPPPPP | 30 |
| VRLPPPVRLPPPVRLPPP | 31 |
| PRPLPPPRPG | 32 |
| SVRRRPRPPYLPRPRPPPFFPPRLPPRIPP | 33 |
| TRSSRAGLQFPVGRVHRLLRK | 34 |
| GIGKFLHSAKKFGKAFVGEIMNS | 35 |
| KWKLFKKIEKVGQNIRDGIIKAGPAVAVVGQATQIAK | 36 |
| ALWMTLLKKVLKAAAKAALNAVLVGANA | 37 |
| GIGAVLKVLTTGLPALISWIKRKRQQ | 38 |
| INLKALAALAKKIL | 39 |
| GFFALIPKIISSPLPKTLLSAVGSALGGSGGQE | 40 |
| LAKWALKQGFAKLKS | 41 |
| SMAQDIISTIGDLVKWIIQTVNXFTKK | 42 |
| LLGDFFRKSKEKIGKEFKRIVQRIKQRIKDFLANLVPRTES | 43 |
| LKKLLKKLLKKLLKKLLKKL | 45 |
| KLKLKLKLKLKLKLKLKL | 46 |
| PAWRKAFRWAWRMLKKAA | 47 |

Peptides may be modified for in vivo use by the addition, at the amino- and/or carboxyl-terminal ends, of a blocking agent to facilitate survival of the peptide in vivo are contemplated. This can be useful in those situations in which the peptide termini tend to be degraded by proteases prior to cellular uptake. Such blocking agents can include, without limitation, additional related or unrelated peptide sequences that can be attached to the amino and/or carboxyl terminal residues of the peptide to be administered. These agents can be added either chemically during the synthesis of the peptide, or by recombinant DNA technology by methods familiar in the art. Alternatively, blocking agents such as pyroglutamic acid or other molecules known in the art can be attached to the amino and/or carboxyl terminal residues. In addition, nanoparticles could be used for the packaging and delivery of the peptide.

1. Synthesis

It will be advantageous to produce peptides using the solid-phase synthetic techniques (Merrifield, 1963). Other peptide synthesis techniques are well known to those of skill in the art (Bodanszky et al., 1976; Peptide Synthesis, 1985; Solid Phase Peptide Synthelia, 1984). Appropriate protective groups for use in such syntheses will be found in the above texts, as well as in Protective Groups in Organic Chemistry, 1973. These synthetic methods involve the sequential addition of one or more amino acid residues or suitable protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group, such as lysine. In addition, vectors that can deliver plasmids can be used to produce the desired peptide, such as in vivo.

Using solid phase synthesis as an example, the protected or derivatized amino acid is attached to an inert solid support through its unprotected carboxyl or amino group. The protecting group of the amino or carboxyl group is then selectively removed and the next amino acid in the sequence having the complementary (amino or carboxyl) group suitably protected is admixed and reacted with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to provide the final peptide. The peptides of the invention are preferably devoid of benzylated or methylbenzylated amino acids. Such protecting group moieties may be used in the course of synthesis, but they are removed before the peptides are used. Additional reactions may be necessary, as described elsewhere, to form intramolecular linkages to restrain conformation.

Aside from the twenty standard amino acids can be used, there are a vast number of “non-standard” amino acids. Two of these can be specified by the genetic code, but are rather rare in proteins. Selenocysteine is incorporated into some proteins at a UGA codon, which is normally a stop codon. Pyrrolysine is used by some methanogenic archaea in enzymes that they use to produce methane. It is coded for with the codon UAG. Examples of non-standard amino acids that are not found in proteins include lanthionine, 2-aminoisobutyric acid, dehydroalanine and the neurotransmitter gamma-aminobutyric acid. Non-standard amino acids often occur as intermediates in the metabolic pathways for standard amino acids—for example ornithine and citrulline occur in the urea cycle, part of amino acid catabolism. Non-standard amino acids are usually formed through modifications to standard amino acids. For example, homocysteine is formed through the transsulfuration pathway or by the demethylation of methionine via the intermediate metabolite S-adenosyl methionine, while hydroxyproline is made by a posttranslational modification of proline.

2. Linkers

Linkers or cross-linking agents may be used to fuse peptides to other proteinaceous sequences. Bifunctional cross-linking reagents have been extensively used for a variety of purposes including preparation of affinity matrices, modification and stabilization of diverse structures, identification of ligand and receptor binding sites, and structural studies. Homobifunctional reagents that carry two identical functional groups proved to be highly efficient in inducing cross-linking between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino-, sulfhydryl-, guanidino-, indole-, or carboxyl-specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

In another example, heterobifunctional cross-linking reagents and methods of using the cross-linking reagents are described in U.S. Pat. No. 5,889,155, specifically incorporated herein by reference in its entirety. The cross-linking reagents combine a nucleophilic hydrazide residue with an electrophilic maleimide residue, allowing coupling in one example, of aldehydes to free thiols. The cross-linking reagent can be modified to cross-link various functional groups and is thus useful for cross-linking polypeptides. In instances where a particular peptide does not contain a residue amenable for a given cross-linking reagent in its native sequence, conservative genetic or synthetic amino acid changes in the primary sequence can be utilized.

3. Mimetics

In addition to the peptides disclosed herein, the present disclosure also contemplates that structurally similar compounds may be formulated to mimic the key portions of peptide or polypeptides of the present disclosure. Such compounds, which may be termed peptidomimetics, may be used in the same manner as the peptides of the present disclosure and, hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Methods for generating specific structures have been disclosed in the art. For example, α-helix mimetics are disclosed in U.S. Pat. Nos. 5,446,128; 5,710,245; 5,840,833; and 5,859,184. Methods for generating conformationally restricted n-turns and n-bulges are described, for example, in U.S. Pat. Nos. 5,440,013; 5,618,914; and 5,670,155. Other types of mimetic turns include reverse and γ-turns. Reverse turn mimetics are disclosed in U.S. Pat. Nos. 5,475,085 and 5,929,237, and γ-turn mimetics are described in U.S. Pat. Nos. 5,672,681 and 5,674,976.

By “molecular modeling” is meant quantitative and/or qualitative analysis of the structure and function of protein-protein physical interaction based on three-dimensional structural information and protein-protein interaction models. This includes conventional numeric-based molecular dynamic and energy minimization models, interactive computer graphic models, modified molecular mechanics models, distance geometry and other structure-based constraint models. Molecular modeling typically is performed using a computer and may be further optimized using known methods. Computer programs that use X-ray crystallography data are particularly useful for designing such compounds. Programs such as RasMol, for example, can be used to generate three dimensional models. Computer programs such as INSIGHT (Accelrys, Burlington, Mass.), GRASP (Anthony Nicholls, Columbia University), Dock (Molecular Design Institute, University of California at San Francisco), and Auto-Dock (Accelrys) allow for further manipulation and the ability to introduce new structures. The methods can involve the additional step of outputting to an output device a model of the 3-D structure of the compound. In addition, the 3-D data of candidate compounds can be compared to a computer database of, for example, 3-D structures.

Compounds of the present disclosure also may be interactively designed from structural information of the compounds described herein using other structure-based design/modeling techniques (see, e.g., Jackson, 1997; Jones et al., 1996). Candidate compounds can then be tested in standard assays familiar to those skilled in the art. Exemplary assays are described herein.

The 3-D structure of biological macromolecules (e.g., proteins, nucleic acids, carbohydrates, and lipids) can be determined from data obtained by a variety of methodologies. These methodologies, which have been applied most effectively to the assessment of the 3-D structure of proteins, include: (a) x-ray crystallography; (b) nuclear magnetic resonance (NMR) spectroscopy; (c) analysis of physical distance constraints formed between defined sites on a macromolecule, e.g., intramolecular chemical crosslinks between residues on a protein (e.g., PCT/US00/14667, the disclosure of which is incorporated herein by reference in its entirety), and (d) molecular modeling methods based on a knowledge of the primary structure of a protein of interest, e.g., homology modeling techniques, threading algorithms, or ab initio structure modeling using computer programs such as MONSSTER (Modeling Of New Structures from Secondary and Tertiary Restraints) (see, e.g., International Application No. PCT/US99/11913, the disclosure of which is incorporated herein by reference in its entirety). Other molecular modeling techniques may also be employed in accordance with this invention (e.g., Cohen et al., 1990; Navia et al., 1992), the disclosures of which are incorporated herein by reference in their entirety). All these methods produce data that are amenable to computer analysis. Other spectroscopic methods that can also be useful in the method of the invention, but that do not currently provide atomic level structural detail about biomolecules, include circular dichroism and fluorescence and ultraviolet/visible light absorbance spectroscopy. One method of analysis is x-ray crystallography.

4. Stabilized Peptides

A particular modification is in the context of peptides as therapeutics is the so-called “Stapled Peptide” technology of Aileron Therapeutics. The general approach for “stapling” a peptide is that two key residues within the peptide are modified by attachment of linkers through the amino acid side chains. Once synthesized, the linkers are connected through a catalyst, thereby creating a bridge that physically constrains the peptide into its native α-helical shape. In addition to helping retain the native structure needed to interact with a target molecule, this conformation also provides stability against peptidases as well as promotes cell-permeating properties.

More particularly, the term “peptide stapling” may encompasses the joining of two double bond-containing sidechains, two triple bond-containing sidechains, or one double bond-containing and one triple bond-containing side chain, which may be present in a polypeptide chain, using any number of reaction conditions and/or catalysts to facilitate such a reaction, to provide a singly “stapled” polypeptide. In a specific embodiment, the introduction of a staple entails a modification of standard peptide synthesis, with α-methy, α-alkenyl amino acids being introduced at two positions along the peptide chain, separated by either three or six intervening residues (i+4 or i+7). These spacings place the stapling amino acids on the same face of the α-helix, straddling either one (i+4) or two (i+7) helical turns. The fully elongated, resin-bound peptide can be exposed to a ruthenium catalyst that promotes cross-linking of the alkenyl chains through olefin metathesis, thereby forming an all-hydrocarbon macrocyclic cross-link. U.S. Pat. Nos. 7,192,713 and 7,183,059, and U.S. Patent Publications 2005/02506890 and 2006/0008848, describing this technology, are hereby incorporated by reference. See also Schafmeister et al., Journal of the American Chemical Society, 122(24): p. 5891-5892 (2000); Walensky et al., Science 305:1466-1470 (2004). Additionally, the term “peptide stitching” refers to multiple and tandem “stapling” events in a single peptide chain to provide a “stitched” (multiply stapled) polypeptide, each of which is incorporated herein by reference. See WO 2008/121767 for a specific example of stitched peptide technology.

5. Peptide Delivery

A nucleic acid encoding a peptide of the present disclosure may be made by any technique known to one of ordinary skill in the art. Non-limiting examples of a synthetic nucleic acid, particularly a synthetic oligonucleotide, include a nucleic acid made by in vitro chemical synthesis using phosphotriester, phosphite or phosphoramidite chemistry and solid phase techniques such as described in EP 266,032, or via deoxynucleoside H-phosphonate intermediates as described by Froehler et al., 1986, and U.S. Pat. No. 5,705,629. A non-limiting example of enzymatically produced nucleic acid includes one produced by enzymes in amplification reactions such as PCR™ (see for example, U.S. Pat. Nos. 4,683,202 and 4,682,195), or the synthesis of oligonucleotides described in U.S. Pat. No. 5,645,897. A non-limiting example of a biologically produced nucleic acid includes recombinant nucleic acid production in living cells, such as recombinant DNA vector production in bacteria (see for example, Sambrook et al. 1989).

The nucleic acid(s), regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). The overall length may vary considerably between nucleic acid constructs. Thus, a nucleic acid segment of almost any length may be employed, with the total length preferably being limited by the ease of preparation or use in the intended recombinant nucleic acid protocol.

a. Nucleic Acid Delivery by Expression Vector

Vectors provided herein are designed, primarily, to express an α2δ-1 C-terminal domain mimetic under the control of regulated eukaryotic promoters (i.e., constitutive, inducible, repressable, tissue-specific). Also, the vectors may contain a selectable marker if, for no other reason, to facilitate their manipulation in vitro.

One of skill in the art would be well-equipped to construct a vector through standard recombinant techniques (see, for example, Sambrook et al., 2001 and Ausubel et al., 1996, both incorporated herein by reference). Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

Viral vectors encoding a α2δ-1 C-terminal domain mimetic may be provided in certain aspects of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor-mediated endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

(i) Adenoviral Vector

One method for delivery of α2δ-1 C-terminal domain mimetic involves the use of an adenovirus expression vector. Although adenovirus vectors are known to have a low capacity for integration into genomic DNA, this feature is counterbalanced by the high efficiency of gene transfer afforded by these vectors. Adenovirus expression vectors include constructs containing adenovirus sequences sufficient to (a) support packaging of the construct and (b) to ultimately express a recombinant gene construct that has been cloned therein.

Adenovirus growth and manipulation is known to those of skill in the art, and exhibits broad host range in vitro and in vivo. This group of viruses can be obtained in high titers, e.g., 109-1011 plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them particular mRNA's for translation.

A recombinant adenovirus provided herein can be generated from homologous recombination between a shuttle vector and provirus vector. Due to the possible recombination between two proviral vectors, wild-type adenovirus may be generated from this process. Therefore, a single clone of virus is isolated from an individual plaque and its genomic structure is examined.

The adenovirus vector may be replication competent, replication defective, or conditionally defective, the nature of the adenovirus vector is not believed to be crucial to the successful practice of the invention. The adenovirus may be of any of the 42 different known serotypes or subgroups A-F. Adenovirus type 5 of subgroup C is the particular starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector. However, other serotypes of adenovirus may be similarly utilized.

Nucleic acids can be introduced to adenoviral vectors as a position from which a coding sequence has been removed. For example, a replication defective adenoviral vector can have the E1-coding sequences removed. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Generation and propagation of replication deficient adenovirus vectors can be performed with helper cell lines. One unique helper cell line, designated 293, was transformed from human embryonic kidney cells by Ad5 DNA fragments and constitutively expresses E1 proteins (Graham et al., 1977). Since the E3 region is dispensable from the adenovirus genome (Jones and Shenk, 1978), adenovirus vectors, with the help of 293 cells, carry foreign DNA in either the E1, the E3, or both regions (Graham and Prevec, 1991).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, a particular helper cell line is 293.

Methods for producing recombinant adenovirus are known in the art, such as U.S. Pat. No. 6,740,320, incorporated herein by reference. Also, Racher et al. (1995) have disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/1) are employed as follows. A cell inoculum, resuspended in 5 ml of medium, is added to the carrier (50 ml) in a 250 ml Erlenmeyer flask and left stationary, with occasional agitation, for 1 to 4 hours. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 hours.

(i) Retroviral Vector

Additionally, the α2δ-1 C-terminal domain mimetic may be encoded by a retroviral vector. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, packaging cell lines are available that should greatly decrease the likelihood of recombination (Markowitz et al., 1988; Hersdorffer et al., 1990).

(i) Adeno-Associated Viral Vector

Adeno-associated virus (AAV) is an attractive vector system for use in the present disclosure as it has a high frequency of integration and it can infect nondividing cells, thus making it useful for delivery of genes into mammalian cells (Muzyczka, 1992). AAV has a broad host range for infectivity (Tratschin, et al., 1984; Laughlin, et al., 1986; Lebkowski, et al., 1988; McLaughlin, et al., 1988), which means it is applicable for use with the present invention. Details concerning the generation and use of rAAV vectors are described in U.S. Pat. Nos. 5,139,941 and 4,797,368.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild-type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte et al., 1995).

6. Other Viral Vectors

Other viral vectors may be employed as constructs in the present disclosure. Vectors derived from viruses such as vaccinia virus (Ridgeway, 1988; Baichwal and Sugden, 1986; Coupar et al., 1988) and herpesviruses may be employed. They offer several attractive features for various mammalian cells (Friedmann, 1989; Ridgeway, 1988; Baichwal and Sugden, 1986; Coupar et al., 1988; Horwich et al., 1990).

A molecularly cloned strain of Venezuelan equine encephalitis (VEE) virus has been genetically refined as a replication competent vaccine vector for the expression of heterologous viral proteins (Davis et al., 1996). Studies have demonstrated that VEE infection stimulates potent CTL responses and has been suggested that VEE may be an extremely useful vector for immunizations (Caley et al., 1997).

In further embodiments, the nucleic acid encoding chimeric CD154 is housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

For example, targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

7. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present invention includes constitutive, inducible, and tissue-specific promoters.

a. Promoter/Enhancers

The expression constructs provided herein comprise a promoter to drive expression of the α2δ-1 C-terminal domain mimetic. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter (Ng, 1989; Quitsche et al., 1989), GADPH promoter (Alexander et al., 1988, Ercolani et al., 1988), metallothionein promoter (Karin et al., 1989; Richards et al., 1984); and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present invention.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

b. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

Additionally, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the F2A (Foot-and-mouth disease virus 2A) or a “2A-like” sequence (e.g., Thosea asigna virus 2A; T2A) (Minskaia and Ryan, 2013).

c. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

8. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

9. Other Methods of Nucleic Acid Delivery

In addition to viral delivery of the nucleic acids encoding α2δ-1 C-terminal domain mimetic, the following are additional methods of recombinant gene delivery to a given host cell and are thus considered in the present disclosure. Thus, other forms of gene therapy may be combined with the therapeutic viral compositions including gene editing methods such as meganucleases, zinc finger nucleases (ZFNs), transcription activator-like effector-based nucleases (TALEN), and the CRISPR-Cas system.

Introduction of a nucleic acid, such as DNA or RNA, may use any suitable methods for nucleic acid delivery for transformation of a cell, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

a. Electroporation

In certain particular embodiments of the present disclosure, the gene construct is introduced into target hyperproliferative cells via electroporation. Electroporation involves the exposure of cells (or tissues) and DNA (or a DNA complex) to a high-voltage electric discharge.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

It is contemplated that electroporation conditions for hyperproliferative cells from different sources may be optimized. One may particularly wish to optimize such parameters as the voltage, the capacitance, the time and the electroporation media composition. The execution of other routine adjustments will be known to those of skill in the art. See e.g., Hoffman, 1999; Heller et al., 1996.

b. Lipid-Mediated Transformation

In a further embodiment, the α2δ-1 C-terminal domain mimetic may be entrapped in a liposome or lipid formulation. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

Lipid-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of lipid-mediated delivery and expression of foreign DNA in cultured chick embryo, HeLa and hepatoma cells.

Lipid based non-viral formulations provide an alternative to adenoviral gene therapies. Although many cell culture studies have documented lipid based non-viral gene transfer, systemic gene delivery via lipid based formulations has been limited. A major limitation of non-viral lipid based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in lipid vehicle stability in the presence and absence of serum proteins. The interaction between lipid vehicles and serum proteins has a dramatic impact on the stability characteristics of lipid vehicles (Yang and Huang, 1997). Cationic lipids attract and bind negatively charged serum proteins. Lipid vehicles associated with serum proteins are either dissolved or taken up by macrophages leading to their removal from circulation. Current in vivo lipid delivery methods use subcutaneous, intradermal, intratumoral, or intracranial injection to avoid the toxicity and stability problems associated with cationic lipids in the circulation. The interaction of lipid vehicles and plasma proteins is responsible for the disparity between the efficiency of in vitro (Felgner et al., 1987) and in vivo gene transfer (Zhu el al., 1993; Philip et al., 1993; Solodin et al., 1995; Liu et al., 1995; Thierry et al., 1995; Tsukamoto et al., 1995; Aksentijevich et al., 1996).

Advances in lipid formulations have improved the efficiency of gene transfer in vivo (Templeton et al. 1997; WO 98/07408). A novel lipid formulation composed of an equimolar ratio of 1,2-bis(oleoyloxy)-3-(trimethyl ammonio)propane (DOTAP) and cholesterol significantly enhances systemic in vivo gene transfer, approximately 150 fold. The DOTAP:cholesterol lipid formulation forms unique structure termed a “sandwich liposome”. This formulation is reported to “sandwich” DNA between an invaginated bi-layer or ‘vase’ structure. Beneficial characteristics of these lipid structures include a positive p, colloidal stabilization by cholesterol, two dimensional DNA packing and increased serum stability. Patent Application Nos. 60/135,818 and 60/133,116 discuss formulations that may be used with the present invention.

The production of lipid formulations often is accomplished by sonication or serial extrusion of liposomal mixtures after (I) reverse phase evaporation (II) dehydration-rehydration (III) detergent dialysis and (IV) thin film hydration. Once manufactured, lipid structures can be used to encapsulate compounds that are toxic (chemotherapeutics) or labile (nucleic acids) when in circulation. Lipid encapsulation has resulted in a lower toxicity and a longer serum half-life for such compounds (Gabizon et al., 1990). Numerous disease treatments are using lipid based gene transfer strategies to enhance conventional or establish novel therapies, in particular therapies for treating hyperproliferative diseases.

B. Thrombin Inhibitors

In some embodiments, GARP cleavage is inhibited by administration of a direct thrombin inhibitor (DTI). DTIs are a class of medication that act as anticoagulants (delaying blood clotting) by directly inhibiting the enzyme thrombin. In another exemplary embodiment, the DTI is univalent. In another exemplary embodiment, the DTI is bivalent. In an exemplary embodiment, the DTI is a member selected from hirudin, bivalirudin (IV), lepirudin, desirudin, argatroban (IV), dabigatran, dabigatran etexilate (oral formulation), melagatran, ximelagatran (oral formulation but liver complications) and prodrugs thereof. In particular aspects, the DTI is dabigatran etexilate.

In particular aspects, the direct thrombin inhibitor is selected from dabigatran or dabigatran etexilate, and the tautomers, racemates, enantiomers, diastereomers, pharmacologically acceptable acid addition salts, solvates, hydrates and prodrugs thereof.

The direct thrombin inhibitor, optionally used in form of its pharmaceutically acceptable acid addition salts, may be incorporated into the conventional pharmaceutical preparation in solid, liquid or spray form. The composition may, for example, be presented in a form suitable for oral, topical, lingual, rectal, parenteral administration or for nasal inhalation: preferred forms includes for example, capsules, tablets, coated tablets, ampoules, suppositories and nasal spray.

### III. METHODS OF TREATMENT

Certain aspects of the present embodiments can be used to prevent or treat a disease or disorder associated with GARP signaling. Signaling of GARP may be reduced by any suitable drugs to prevent cancer cell proliferation. Preferably, such substances would be an inhibitor of GARP cleavage, such as a GARP peptide provided herein which prevents thrombin binding and/or a direct thrombin inhibitor. In further embodiments, there are provided methods of identifying platelet activation by detecting an increase in soluble GARP, and optionally latent TGFβ, such as by ELISA. A subject identified to have platelet activation would be administered an anti-platelet agent, such as a direct thrombin inhibitor, and/or a GARP peptide.

Provided herein, in certain embodiments, are methods for treating or delaying progression of cancer in an individual comprising administering to the individual an effective amount an an anti-platelet agent, such as a direct thrombin inhibitor, and/or a GARP peptide. Examples of cancers contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer.

In some embodiments, the individual has cancer that is resistant (has been demonstrated to be resistant) to one or more anti-cancer therapies. In some embodiments, resistance to anti-cancer therapy includes recurrence of cancer or refractory cancer. Recurrence may refer to the reappearance of cancer, in the original site or a new site, after treatment. In some embodiments, resistance to anti-cancer therapy includes progression of the cancer during treatment with the anti-cancer therapy. In some embodiments, the cancer is at early stage or at late stage.

In some embodiments of the methods of the present disclosure, activated CD4 and/or CD8 T cells in the individual are characterized by γ-IFN producing CD4 and/or CD8 T cells and/or enhanced cytolytic activity relative to prior to the administration of the combination. γ-IFN may be measured by any means known in the art, including, e.g., intracellular cytokine staining (ICS) involving cell fixation, permeabilization, and staining with an antibody against γ-IFN. Cytolytic activity may be measured by any means known in the art, e.g., using a cell killing assay with mixed effector and target cells.

A an anti-platelet agent, such as a direct thrombin inhibitor, and/or a GARP peptide may be administered before, during, after, or in various combinations relative to an immunotherapy, such as an immune checkpoint inhibitor. The administrations may be in intervals ranging from concurrently to minutes to days to weeks. In embodiments where the anti-platelet agent, such as a direct thrombin inhibitor, and/or a GARP peptide is provided to a patient separately from an immunotherapy, one would generally ensure that a significant period of time did not expire between the time of each delivery, such that the two compounds would still be able to exert an advantageously combined effect on the patient. In such instances, it is contemplated that one may provide a patient with the first therapy and the second therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

The anti-platelet agent, such as a direct thrombin inhibitor, and/or a GARP peptide and immunotherapy may be administered by the same route of administration or by different routes of administration. In some embodiments, the an anti-platelet agent, such as a direct thrombin inhibitor, and/or a GARP peptide is administered intravenously, intramuscularly, subcutaneously, topically, orally, transdermally, intraperitoneally, intraorbitally, by implantation, by inhalation, intrathecally, intraventricularly, or intranasally. An effective amount of the therapy may be administered for prevention or treatment of disease. The appropriate dosage of the therapy may be determined based on the type of disease to be treated, severity and course of the disease, the clinical condition of the individual, the individual's clinical history and response to the treatment, and the discretion of the attending physician.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (in particular 10 ml), while for tumors of <4 cm, a volume of about 1-3 ml will be used (in particular 3 ml). Multiple injections delivered as single dose comprise about 0.1 to about 0.5 ml volumes.

B. Pharmaceutical Compositions

Where clinical application of a therapeutic composition containing an inhibitory antibody is undertaken, it will generally be beneficial to prepare a pharmaceutical or therapeutic composition appropriate for the intended application. In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein.

Also provided herein are pharmaceutical compositions and formulations comprising an anti-platelet agent, such as a direct thrombin inhibitor, a GARP peptide, and/or an immunotherapy and a pharmaceutically acceptable carrier.

The therapeutic compositions of the present embodiments are advantageously administered in the form of injectable compositions either as liquid solutions or suspensions; solid forms suitable for solution in, or suspension in, liquid prior to injection may also be prepared. These preparations also may be emulsified.

The active compounds can be formulated for parenteral administration, e.g., formulated for injection via the intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22nd edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

C. Anti-Platelet Agents

Certain embodiments of the present methods concern anti-platelet agents. The phrase “anti-platelet agent” refers to any compound which inhibits activation, aggregation, and/or adhesion of platelets, and is intended to include all pharmaceutically acceptable salts, prodrugs e.g., esters and solvate forms, including hydrates, of compounds which have the activity, compounds having one or more chiral centers may occur as racemates, racemic mixtures and as individual diastereomers or enantiomers with all such isomeric forms and mixtures thereof being included, any crystalline polymorphs, co-crystals and the amorphous form are intended to be included.

Non-limiting examples of antiplatelet agents that may be used in the oral dosage forms of the present disclosure include adenosine diphosphate (ADP) antagonists or P₂Yi₂ antagonists, phosphodiesterase (PDE) inhibitors, adenosine reuptake inhibitors, Vitamin K antagonists, heparin, heparin analogs, direct thrombin inhibitors, glycoprotein IIB/IIIA inhibitors, anti-clotting enzymes, as well as pharmaceutically acceptable salts, isomers, enantiomers, polymorphic crystal forms including the amorphous form, solvates, hydrates, co-crystals, complexes, active metabolites, active derivatives and modifications, pro-drugs thereof, and the like.

ADP antagonists or P₂Y₁₂ antagonists block the ADP receptor on platelet cell membranes. This P₂Yi₂ receptor is important in platelet aggregation, the cross-linking of platelets by fibrin. The blockade of this receptor inhibits platelet aggregation by blocking activation of the glycoprotein Ilb/IIIa pathway. In an exemplary embodiment, the antiplatelet agent is an ADP antagonist or P₂Yi₂ antagonist. In another exemplary embodiment, the antiplatelet agent is a thienopyridine. In another exemplary embodiment, the ADP antagonist or P₂Yi₂ antagonist is a thienopyridine.

In another exemplary embodiment, the ADP antagonist or P₂Yi₂ antagonist is a member selected from sulfinpyrazone, ticlopidine, clopidogrel, prasugrel, R-99224 (an active metabolite of prasugrel, supplied by Sankyo), R-1381727, R-125690 (Lilly), C-1330-7, C-50547 (Millennium Pharmaceuticals), INS-48821, INS-48824, INS-446056, INS-46060, INS-49162, INS-49266, INS-50589 (Inspire Pharmaceuticals) and Sch-572423 (Schering Plough). In another exemplary embodiment, the ADP antagonist or P₂Yi₂ antagonist is ticlopidine hydrochloride (TICLID™). In another exemplary embodiment, the ADP antagonist or P₂Yi₂ antagonist is a member selected from sulfinpyrazone, ticlopidine, AZD6140, clopidogrel, prasugrel and mixtures thereof. In another exemplary embodiment, the ADP antagonist or P₂Yi₂ antagonist is clopidogrel. In another exemplary embodiment, the therapeutically effective amount of clopidogrel is from about 50 mg to about 100 mg. In another exemplary embodiment, the therapeutically effective amount of clopidogrel is from about 65 mg to about 80 mg. In another exemplary embodiment, the ADP antagonist or P₂Yi₂ antagonist is a member selected from clopidogrel bisulfate (PLA VIX™), clopidogrel hydrogen sulphate, clopidogrel hydrobromide, clopidogrel mesylate, cangrelor tetrasodium (AR-09931 MX), ARL67085, AR-C66096 AR-C 126532, and AZD-6140 (AstraZeneca). In another exemplary embodiment, the ADP antagonist or P₂Yi₂ antagonist is prasugrel. In another exemplary embodiment, the therapeutically effective amount of prasugrel is from about 1 mg to about 20 mg. In another exemplary embodiment, the therapeutically effective amount of clopidogrel is from about 4 mg to about 11 mg. In another exemplary embodiment, the ADP antagonist or P₂Yi₂ antagonist is a member selected from clopidogrel, ticlopidine, sulfinpyrazone, AZD6140, prasugrel and mixtures thereof.

In certain embodiments the anti-platelet agent is clopidogrel or a pharmaceutically acceptable salt, solvate, polymorph, co-crystal, hydrate, enantiomer or prodrug thereof. In another embodiment clopidogrel or pharmaceutically acceptable salt, solvate, polymorph, co-crystal, hydrate, enantiomer or prodrug thereof is a powder.

A PDE inhibitor is a drug that blocks one or more of the five subtypes of the enzyme phosphodiesterase (PDE), preventing the inactivation of the intracellular second messengers, cyclic adenosine monophosphate (cAMP) and cyclic guanosine monophosphate (cGMP), by the respective PDE subtype(s). In an exemplary embodiment, the antiplatelet agent is a PDE inhibitor. In an exemplary embodiment, the antiplatelet agent is a selective cAMP PDE inhibitor, hi an exemplary embodiment, the PDE inhibitor is cilostazol (Pletal™).

Adenosine reuptake inhibitors prevent the cellular reuptake of adenosine into platelets, red blood cells and endothelial cells, leading to increased extracellular concentrations of adenosine. These compounds inhibit platelet aggregation and cause vasodilation, hi an exemplary embodiment, the antiplatelet agent is an adenosine reuptake inhibitor. In an exemplary embodiment, the adenosine reuptake inhibitor is dipyridamole (Persantine™).

Vitamin K inhibitors are given to people to stop thrombosis (blood clotting inappropriately in the blood vessels). This is useful in primary and secondary prevention of deep vein thrombosis, pulmonary embolism, myocardial infarctions and strokes in those who are predisposed. In an exemplary embodiment, the anti-platelet agent is a Vitamin K inhibitor, hi an exemplary embodiment, the Vitamin K inhibitor is a member selected from acenocoumarol, clorindione, dicumarol (Dicoumarol), diphenadione, ethyl biscoumacetate, phenprocoumon, phenindione, tioclomarol and warfarin.

Heparin is a biological substance, usually made from pig intestines. It works by activating antithrombin III, which blocks thrombin from clotting blood. In an exemplary embodiment, the antiplatelet agent is heparin or a prodrug of heparin. In an exemplary embodiment, the antiplatelet agent is a heparin analog or a prodrug of a heparin analog. In an exemplary embodiment, the heparin analog a member selected from Antithrombin III, Bemiparin, Dalteparin, Danaparoid, Enoxaparin, Fondaparinux (subcutaneous), Nadroparin, Parnaparin, Reviparin, Sulodexide, and Tinzaparin.

Direct thrombin inhibitors (DTIs) are a class of medication that act as anticoagulants (delaying blood clotting) by directly inhibiting the enzyme thrombin. In an exemplary embodiment, the antiplatelet agent is a DTI. In another exemplary embodiment, the DTI is univalent. In another exemplary embodiment, the DTI is bivalent. In an exemplary embodiment, the DTI is a member selected from hirudin, bivalirudin (IV), lepirudin, desirudin, argatroban (IV), dabigatran, dabigatran etexilate (oral formulation), melagatran, ximelagatran (oral formulation but liver complications) and prodrugs thereof.

In an exemplary embodiment, the anti-platelet agent is a member selected from aloxiprin, beraprost, carbasalate calcium, cloricromen, defibrotide, ditazole, epoprostenol, indobufen, iloprost, picotamide, rivaroxaban (oral FXa inhibitor) treprostinil, triflusal, or prodrugs thereof.

D. Additional Therapy

In certain embodiments, the compositions and methods of the present embodiments involve a direct thrombin inhibitor and/or a GARP peptide, in combination with a second or additional therapy. Such therapy can be applied in the treatment of any disease that is associated with GARP-mediated cell proliferation. For example, the disease may be cancer.

In certain embodiments, the compositions and methods of the present embodiments involve a a direct thrombin inhibitor and/or a GARP peptide in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with both an antibody or antibody fragment and a second therapy. A tissue, tumor, or cell can be contacted with one or more compositions or pharmacological formulation(s) comprising one or more of the agents (i.e., antibody or antibody fragment or an anti-cancer agent), or by contacting the tissue, tumor, and/or cell with two or more distinct compositions or formulations, wherein one composition provides 1) an antibody or antibody fragment, 2) an anti-cancer agent, or 3) both an antibody or antibody fragment and an anti-cancer agent. Also, it is contemplated that such a combination therapy can be used in conjunction with chemotherapy, radiotherapy, surgical therapy, or immunotherapy.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

An inhibitory antibody may be administered before, during, after, or in various combinations relative to an anti-cancer treatment. The administrations may be in intervals ranging from concurrently to minutes to days to weeks. In embodiments where the antibody or antibody fragment is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

Various combinations may be employed. For the example below a direct thrombin inhibitor and/or a GARP peptide, is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegaIl); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen (Carter et al., 2008; Teicher 2014; Leal et al., 2014). Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach (Teicher 2009) and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints are molecules in the immune system that either turn up a signal (e.g., co-stimulatory molecules) or turn down a signal. Inhibitory checkpoint molecules that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008.449, all incorporated herein by reference. Other PD-1 axis antagonists for use in the methods provided herein are known in the art such as described in U.S. Patent Pub. Nos. 20140294898, 2014022021, and 20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO01/14424, WO98/42752; WO00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WOO 1/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesions such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IV. ARTICLES OF MANUFACTURE OR KITS

In various aspects of the embodiments, a kit is envisioned containing therapeutic agents and/or other therapeutic and delivery agents. In some embodiments, the present embodiments contemplates a kit for preparing and/or administering a therapy of the embodiments. The kit may comprise one or more sealed vials containing any of the pharmaceutical compositions of the present embodiments. The kit may include, for example, at least one GARP antibody as well as reagents to prepare, formulate, and/or administer the components of the embodiments or perform one or more steps of the inventive methods. In some embodiments, the kit may also comprise a suitable container, which is a container that will not react with components of the kit, such as an eppendorf tube, an assay plate, a syringe, a bottle, or a tube. The container may be made from sterilizable materials such as plastic or glass.

In some embodiment, an article of manufacture or a kit is provided comprising adoptive T cells and an anti-platelet agent (e.g., anti-GARP antibody) is also provided herein. The article of manufacture or kit can further comprise a package insert comprising instructions for using the adoptive T cells in conjunction with an anti-platelet agent to treat or delay progression of cancer in an individual or to enhance immune function of an individual having cancer. Any of the adoptive T cells and/or anti-platelet agents described herein may be included in the article of manufacture or kits. In some embodiments, the adoptive T cells and anti-platelet agent are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Role of GARP Expression on Platelets

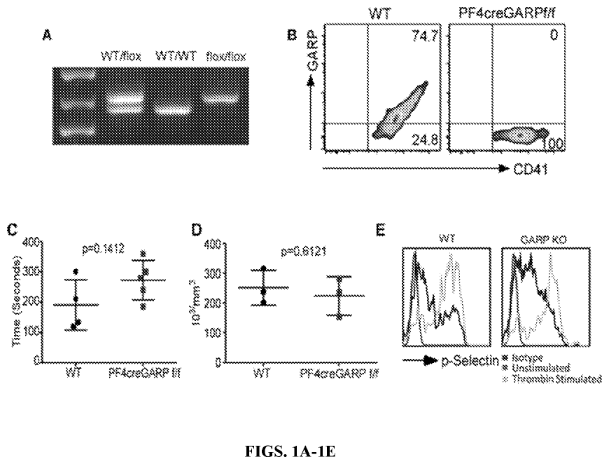

GARP deletion on platelets does not alter platelet activation and number: For the generation of mice with specific deletion of GARP on platelets the Cre-Lox recombination system was employed. Pf4creGARPf/f mice and their littermates were obtained by crossing GARP flox/flox with Pf4cre mice (Edwards et al., 2013; Tiedt et al., 2007). PCR analysis of genomic DNA demonstrated the deletion of Exonl in both alleles of GARP gene in Pf4creGARPf/f mice (FIG. 1A). Phenotypical characterization of WT and Pf4creGARPf/f mice was performed by flow-cytometry analysis of GARP expression on CD41+ activated platelets. In Pf4creGARPf/f mice, GARP expression on platelets was totally abrogated (FIG. 1B). To assess whether the function of murine platelets was jeopardized by GARP abrogation, a bleeding time test was performed on Pf4creGARPf/f and WT littermates. There was no statistical difference between the two groups (FIG. 1C). This showed that absence of GARP from the platelet surface did not affect the ability of platelets to control hemostasis. In this system, GARP deletion occurs in the megakaryocytes which are precursors of platelets. To determine whether this might affect platelet generation, platelet count was performed, yet a normal platelet number was observed in Pf4creGARPf/f mice (FIG. 1D). Finally, thrombin-mediated platelet activation was evaluated by p-Selectin expression. Upon thrombin ex vivo stimulation both platelet GARP KO and WT upregulated p-Selectin as marker of platelet activation (FIG. 1E). Overall these results indicate that GARP is likely not essential for normal platelet biogenesis and hemostasis.

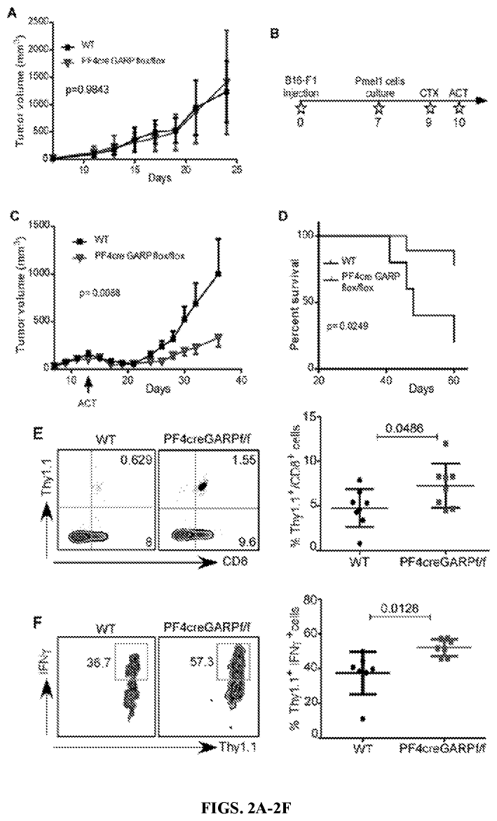

Platelet-derived GARP/TGF-β complex blunts adoptive T cell therapy of melanoma: Although GARP may not have a role in normal platelets number and function, upon thrombin activation GARP is abundantly upregulated on platelets, suggesting that it might play a role in active platelets beside thrombus formation. Platelet-derived TGF-β plays a role in enhancing tumor progression, thus, Pf4creGARPf/f and WT littermates mice were challenged with B16-F1 melanoma cells to investigate whether GARP, by enhancing TGF-β activation, might promote tumor progression. Surprisingly, tumors in both groups grew at the same rate and with the same aggressiveness (FIG. 2A). Knowing that B-16-F1 is a poorly immunogenic tumor, it was asked whether GARP plays negative roles in the tumor microenvironment in anti-tumor T cell immunity. This hypothesis was addressed by comparing the efficacy of adoptive T cell therapy of melanoma in WT and Pf4creGARP f/f recipient mice. B16-F1 melanomas were established in either WT or Pf4creGARP f/f mice, followed by lymphodepletion with Cy on day 9, and the infusion of ex vivo activated Pmel T cells on day 10 (FIG. 2B). Tumors were controlled much more efficiently in Pf4creGARP f/f mice compared with WT mice as represented by the tumor curve growth (FIG. 2C) and survival (FIG. 2D). This was associated with longer persistency (FIG. 2E) and better functionality (FIG. 2F) of adoptively transferred Pmel 1 cells in Pf4creGARP f/f mice peripheral blood. These results indicate that GARP on platelets decreases the efficacy of T cell-mediated anti-tumor immunity.

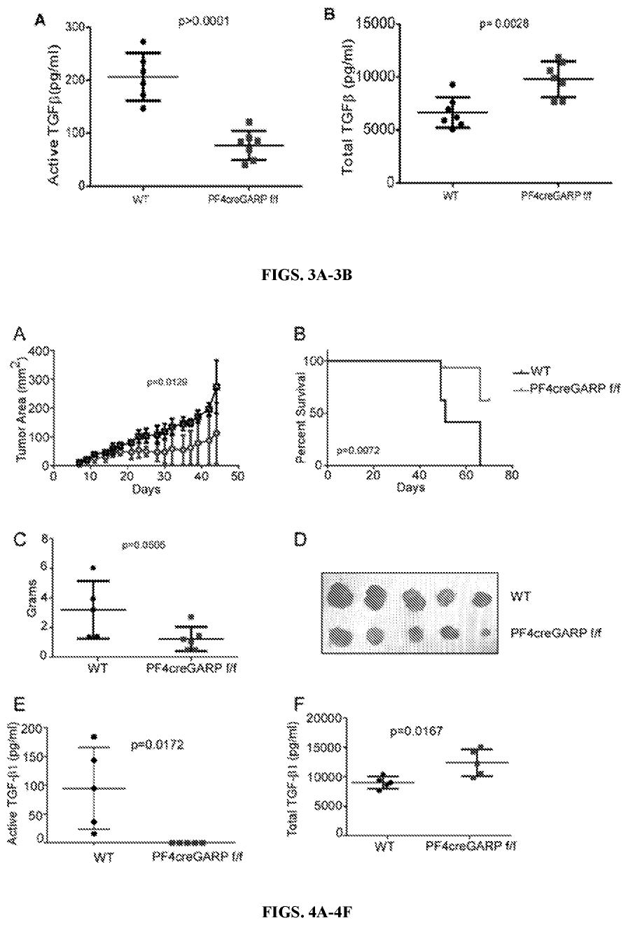

Platelet-intrinsic GARP plays critical roles in generating active TGFβ: Systemic blockade of TGF-β signaling improves the effectiveness of adoptively transferred T cells (Wallace et al., 2008). Given that GARP enhances TGF-β activation and that platelets are the major reservoir of TGF-3, it was decided to investigate TGF-β activation status as a potential mechanism to explain the better anti-tumor activity observed in Pf4creGARPf/f mice. It was observed that KO mice had a severe impairment in the activation of latent-TGF-β since they showed reduced active TGF-β (FIG. 3A) and increased total TGF-β (FIG. 3B).

Targeting platelet-derived GARP/TGF-β complex improves MC38 tumor control: To further investigate the TGF-β driven phenotype that was observed using the B16-F1 tumor model, the MC38 colon carcinoma model was employed. In this model, tumor infiltrated CD8⁺ T cell functionality is severely impaired by TGF-β signaling (di Bari et al., 2009). Interestingly the rate of tumor growth was the same in both groups in the first two weeks after tumor injection, and it was only around day 15 that the two curves started to separate showing better tumor growth control by Pf4creGARP f/f mice (FIG. 4A). This might indicate that platelet derived TGF-β has a potent immunosuppressive effect on the adaptive arm of the anti-tumor immunity. This was reflected also by the longer overall survival experienced by Pf4creGARP f/f (FIG. 4B), along with primary tumor weight (FIG. 4C and FIG. 4D). ELISA was used to measure the concentration of serum active and total TGF-β in tumor bearing mice and, there was a large reduction of the mature form of TGF-β (FIG. 4E) in parallel with an increase of the total form of the same cytokine (FIG. 4F).

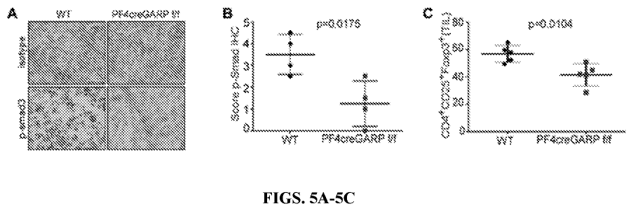

Physiologically, cancer represents a non-healing wound where the coagulation cascade forms a fibrin cloak where platelets are constantly activated (Jurk and Kehrel, 2005). Therefore, it was reasoned that the GARP may have an effect on TGF-β derived platelets in the tumor. For this reason, tumors from WT and Pf4creGARPf/f mice were analyzed for active TGF-β signaling pathway. Smad3 phosphorylation was significantly increased in WT tumor as shown by IHC pictures (FIG. 5A) and score (FIG. 5B). Accordingly, GARP abrogation in platelets impaired regulatory T cell expansion (FIG. 5B).

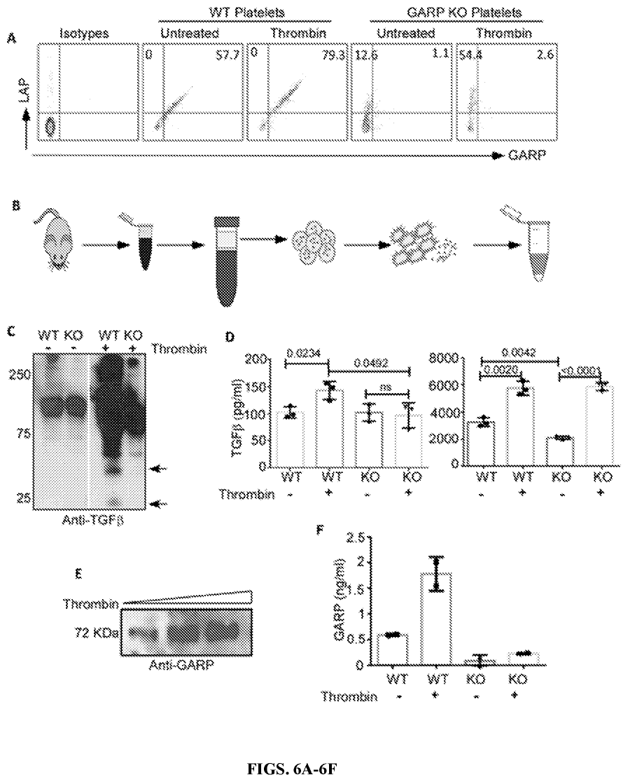

Platelet GARP is increased upon thrombin stimulation and enhances active TGFβ release in Platelets Releasate (PR): In the PR derived from human platelets stimulated with thrombin, TGF-β is one of the most abundant cytokines. To study how platelet GARP modulates TGF-β release and activation in PR, WT and GARP KO platelets were stimulated with thrombin and GARP expression and TGF-β activation status were analyzed. Active platelets increased surface GARP/LAP expression by about 45%. Notably, LAP expression was also increased in activated GARP KO platelets, suggesting the upregulation of other latent TGF-β binding receptors, however the amount of latent TGF-β on GARP KO platelets was still less compared to activate WT platelets (FIG. 6A). Next, WT and GARP KO platelets were isolated from peripheral blood and activated in presence or absence of thrombin (FIG. 6B). Western blot analysis of the resulting PR revealed that upon thrombin stimulation GARP on platelets enhances the release of total as well as active TGF-β. (FIG. 6C). Furthermore TGF-β ELISA performed on the PR confirmed that GARP is critical for activation of the TGF-β released in PR (FIG. 6D, left panel), while the amount of total TGF-β was not affected by the lack of GARP (FIG. 6D, right panel). It was next hypothesized that just like in Tregs, GARP could be also be released from activated platelets as soluble protein in the PR. For this reason murine WT platelets were stimulated with increasing units of mouse thrombin, the PR was collected and analyzed GARP by WB. Strikingly, soluble GARP was observed in the PR in response to thrombin in the dose-dependent fashion (FIG. 6E). Accordingly, further ELISA data showed that GARP was released in PR only upon thrombin mediated platelet activation (FIG. 6F).

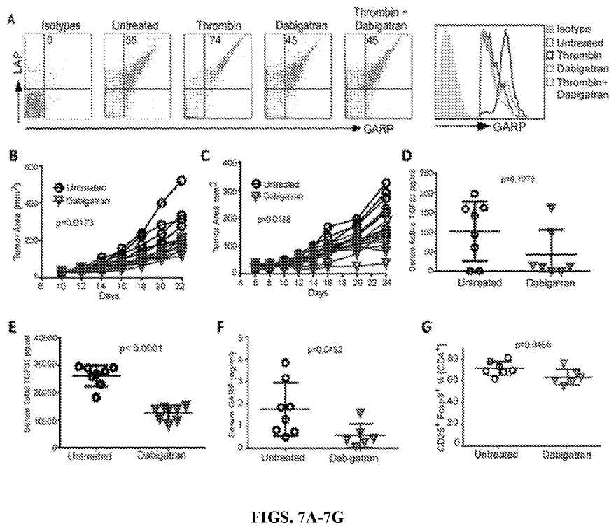

Direct thrombin inhibitor Dabigatran Etexilate reduces platelet GARP expression and protects against melanoma and colon cancer: So far it was demonstrated that thrombin-stimulated platelets release active TGF-β through the mediation of GARP. Now, to establish the clinical relevance of the Thrombin-GARP/TGF-β axis, the direct thrombin inhibitor, Dabigatran Etexilate was employed. Flow cytometry analysis of thrombin activated platelets showed that the increase of GARP expression on platelets can be neutralized by the inhibitory activity of Dabigatran (FIG. 7A). The anti-tumor efficacy of Dabigatran Etexilate was tested in 2 different tumor models. Strikingly, it was observed that Dabigatran reduced B16-F1 tumor aggressiveness even in absence of adoptive T cells transfer (FIG. 7B). The efficacy of Dabigatran was then tested with MC38 colon carcinoma where, as well as B16-F1, the sole direct inhibition of thrombin was sufficient to decrease the rate of tumor growth (FIG. 7C). As a further proof of the mechanism employed by dabigatran to exert its antitumor activity, serum active and total TGF-β, and serum GARP concentrations were assessed by ELISA. Even though not statistically significant the pool of active TGF-β was drastically reduced: the mean observed in untreated mice was 101.3±26.59, versus 42.47±23.73 in Dabigatran treated mice. Similarly, blocking thrombin mediated platelet activation reduced serum total TGF-β (FIG. 7E). Notably, Dabigatran impairs the release of serum soluble GARP from platelets (FIG. 7E) and reduces the TGF-β mediated induction of Tregs in the tumor microenvironment (FIG. 7G).

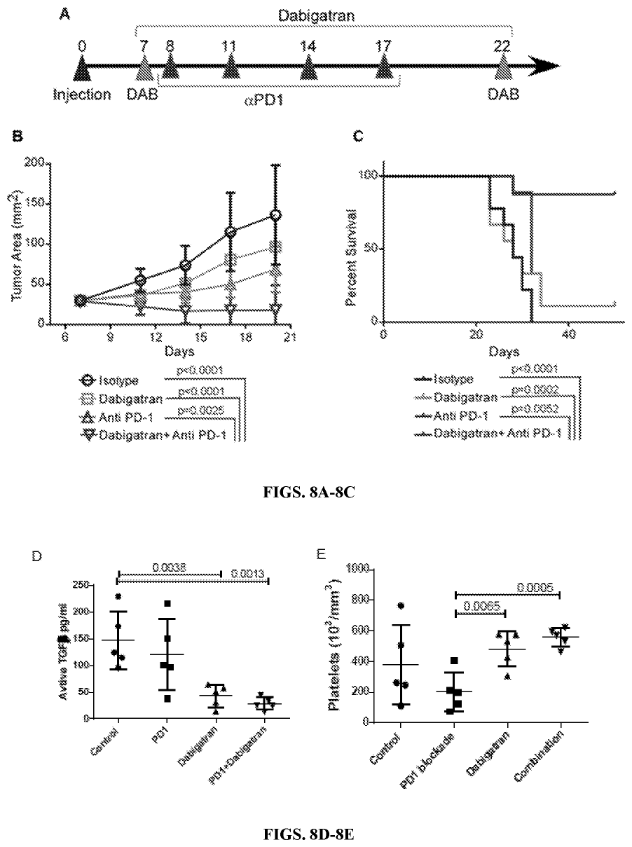

Next, Dabigatran was used in combination with PD1 blockade to block thrombin and consequentially platelet activation to achieve both a reduction of systemic and circulating TGF-β, and the abrogation of platelet-mediated tumor support. Platelets, indeed, secrete many other factors beyond TGF-β that facilitate tumor immune evasion. Animals harboring palpable tumors were daily treated with 3 mg/mouse dabigatran by oral gavage. Additionally 200 μg of anti-PD1 blockade antibody was administered every 3 days starting on day 8 (FIG. 8A). Single therapies alone were equally effective in reducing tumor growth, however mice treated with combination of anti-PD1 and Dabigatran achieved total regression (FIG. 8B) as confirmed by prolonged survival (FIG. 8C)

It was shown that GARP enhances the activation of latent TGF-β released by platelets and in doing so potentiates platelet tumorigenic activity. The present studies also showed for the first time that GARP is released in the PR of thrombin activated platelets. Furthermore, it was demonstrated that the release of mature TGF-β is the last step of a new thrombin-GARP axis that can be pharmacologically blocked by direct thrombin inhibitors. Studies with genetic ablation of GARP from platelets selectively led to a clear conclusion that serum active TGF-β depends on the platelet surface GARP/TGF-β complex. The increased serum latent TGF-β might be explained as a compensatory mechanism operated by platelets failing to generate a mature form of the cytokine. The decreased TGF-β signaling in TME highlighted by the ablation of Smad3 phosphorylation is strong evidence for the functional TGF-β in mice with specific deletion of GARP from platelets.

Mechanistically, the studies demonstrated the presence of a pathway where thrombin enhances GARP/latent TGF-β expression on platelets that in turn results in the release of active TGF-β and GARP in the PR. Not only TGF-β, but soluble GARP also has immunomodulatory function, thus reinforcing the pro-tumorigenic PR activity. Based on these findings, a high concentration of circulating soluble GARP and TGF-β1 could be regarded as valuable biomarkers for sustained platelet activation. Accordingly, high level of serum TGF-β is a poor prognostic factor in several malignancies and plasma soluble GARP was increased in metastatic prostate cancer patients (FIG. 3-8), reinforcing the concept of platelets and cancer bi-directional activation. Based on our results it is reasonable to hypothesize that platelets PR is one of the major contributors to systemic circulating TGF-β and GARP pool in cancer patients. The results from the combination therapy of Dabigatran Etexilate and PD1 blockade support the notion that the efficacy of re-activated tumor-specific T cells can be further reinforced by blocking the immunosuppressive TGF-β-rich platelet clot that protects tumors.

### Example 2—Inhibition of GAPR Cleavage

GARP is cleaved on the cell surface releasing a 29 KDa fragment in the extracellular environment: It was demonstrated that the molecular chaperone gp96 is critical for cell surface expression of GARP and membrane latent TGF-β (Jurk and Kehrel, 2015). To investigate the role of gp96 in the formation of the soluble GARP, GARP was expressed in WT and gp96 KO PreB cells. Cell lysates analysis revealed the presence of three forms of GARP protein: full length protein (72 KD) expressed in both WT and gp96 KD cells, and 2 smaller forms of GARP protein (44 KD and 29 KD) not present in the gp96 KD cells (FIG. 1A). The formation of smaller fragments of GARP only in presence of gp96 supported the idea that GARP might be shed at the cell surface and be released in the extracellular environment. To address this possibility, the presence of GARP was analyzed in cell lysate and conditioned medium from GARP expressing cells. It was observed that the 29 KD fragment was abundantly present in the conditioned medium, and only a small fraction of the protein was present as full length in the extracellular environment (FIG. 1B). Mass spectrometry analysis showed that the 29 KD GARP fragment belongs to part of the N⁺ terminal domain (FIG. 1C).

Surface GARP is cleaved by thrombin: The next question that was asked aimed to define the mechanism of GARP cleavage. First, the roles of furin, several matrix metalloproteinases and serine proteases inhibitors were tested. It was found that the lower GARP fragment decreased only when the serine proteases were inhibited. In parallel, GARP amino acidic sequence was analyzed using the online available resource portal ExPASy to predict the potential proteases cleavage sites. Among the list of enzymes that were suggested to interact with GARP, thrombin attracted attention for two reasons: first, thrombin is a surface serine protease; second, based on the ExPASy prediction, thrombin mediated cleavage generates two GARP fragments of the same molecular weight of the fragment that were already observed, 44 and 29 KD. (FIG. 2A). For these reasons, it was decided to further study the potential role of thrombin in GARP cleavage. It was found that upon treatment with increasing concentration of thrombin, both 44 and 29 KD fragments increased in a dose dependent fashion (FIG. 2B). Since GARP cleavage occurred in the presence and absence of serum in the conditioned medium, it was reasoned that thrombin was produced by the tumor cells. To address this point further, thrombin was knocked down in GARP expressing cells and it was noticed that the 29 KD GARP fragment was almost completely abrogated, while full-length protein staining intensity was increased (FIG. 2C). In Example 1, GARP expression and the function of platelets was analyzed. Just like preB cells, platelet GARP is cleaved by thrombin, revealing the generality of the findings. (FIG. 2D)

GARP upregulates thrombin gene expression: Several cancers upregulate thrombin expression as a survival factor to amplify TCIPA. Also, GARP exerts an oncogenic function in epithelial cells. For these reasons, it was next asked whether GARP expression could upregulate thrombin expression. Interestingly, enforced GARP correlated with enhanced expression of thrombin mRNA (FIG. 3A), suggesting a positive correlation between GARP and thrombin gene transcription. In support of GARP-Thrombin correlation, a positive association (R=0.127) was found between GARP and Thrombin mRNA in a cohort of 59 breast cancer patients (FIG. 3B).

Thrombin cleaves GARP at the amino acid position 267 and 286 between proline and arginine: To prove that thrombin cleaves GARP at the predicted cleavage sites, the two proline-arginine binding sites were mutated at 267-268 and 286-287 amino acidic positions to alanine-alanine. Thus, 3 types of mutants: PR 267-268AA (GARP 267), PR 286-287AA (GARP 286), and double mutant (DM) PR 267-268AA PR 286-287AA (GARP 267-286, -DM) were generated. As GARP cleavage occurs on the cell surface, it was first confirmed that the mutations did not affect surface GARP expression. Flow cytometry analysis showed that GARP harboring 267, 286, and both 267+286 mutations are normally expressed on the cell surface (FIG. 4A). Strikingly, parallel western blot analysis of cell lysates and conditioned media revealed that thrombin indeed cleaves GARP at the predicted cleavage sites (FIG. 4B). Notably, the smaller cleaved GARP fragment from GARP 267 and GARP 286, are reduced in molecular weight and intensity, but still present. The 29KDa fragment from GARP harboring the two mutations 267-286 (GARP-DM) is completely abrogated, indicating that thrombin cleaves GARP at both binding sites (FIG. 4B). Next, it tested whether GARP-DM was resistant to the cleavage of exogenous thrombin. To this end, PreB GARP-WT and PreB GARP-DM were treated with increasing concentrations of thrombin. Both 44 and 29 KD fragments increased in a dose dependent fashion in GARP-WT, no cleaved products were observed in GARP-DM (FIG. 4C). To further confirm thrombin dependent GARP cleavage, a competition assay was performed using a recombinant fragment of GARP containing the two Proline Arginine thrombin binding sites, named T250 (FIG. 4D) In accordance with the previous results, T250 competed with surface GARP in binding with thrombin and was able to reduce GARP cleavage even in presence of exogenous thrombin (FIG. 4E).

GARP expression facilitates cleavage of pro-TGF-β in mature TGF-β and secretion of latent TGF-β. It was hypothesized that thrombin mediated cleavage might be a mechanism to regulate the activation of latent TGF-β bound to GARP. A fundamental prerequisite was the binding of latent TGF-β to mutated GARP, thus it was checked if the mutations affected the ability of GARP to bind latent TGF-β. Surface flow cytometry analyses show that the 3 GARP mutants still retained the ability bind to LAP, and interestingly LAP binding increased in GARP-DM (FIG. 5B). GARP has been shown to enhance pro-TGF-β maturation and to mediate latent-TGF-β secretion from Tregs (Gauthy. et al. 2013). Thus, it was decided to test whether thrombin plays a role in these two GARP's functions. Surprisingly, the inhibition of thrombin-mediated cleavage did not affect the ability of GARP to facilitate pro-TGF-β maturation (FIG. 5B), however it affects the secretion of latent TGF-β in the cell supernatant (FIG. 5C). This result is consistent with the previous finding that thrombin cleavage occurs only on the cell surface and sheds light on the importance of thrombin in releasing latent TGF-β. Accordingly, low total TGF-β in conditioned medium of GARP DM correlates with the higher expression of LAP on the surface as shown by surface flow cytometry analysis (FIG. 5A).

Recombinant GARP protein is bound to latent TGFβ and is cleaved by thrombin: To further study the function of GARP cleavage, a recombinant form of soluble GARP (sGARP) was employed where the transmembrane domain was replaced by IgG1 Fc fragment. The function of the Fc domain was to facilitate the protein purification using a Protein A column system. This recombinant protein mimics the soluble cleaved GARP product. Western blot analysis of the purified sGARP revealed that the recombinant protein was isolated in complex with latent TGF-β. Indeed, non-reducing and non-denaturating SDS-PAGE (ND) shows a large complex that is recognized at the same molecular weight by both anti-GARP and anti-TGF-β antibody. When denaturated and reduced (D), the complex dissociates in sGARP and TGF-β (FIG. 6A). TGF-β ELISA of sGARP indeed showed that at serial dilutions of sGARP corresponded a parallel dilution of total TGF-β (FIG. 6B). Soluble GARP/latent TGF-β complex was analyzed by western blot analysis upon dose dependent treatment with thrombin. As expected, digestion of soluble GARP with thrombin gave rise to 3 fragments: GARP full length (72 KD), and the 2 smaller fragments of 44 and 29 KD (FIG. 6C). More interestingly, in parallel with the formation of cleaved GARP products, latent TGF-β was released as represented by non-reducing conditions western blot analysis (FIG. 6D). No active TGF-β dimer (25KDa) was detected by western Blot analysis. Interestingly, GARP cleavage and latent TGF-β occurs occurred in parallel, suggesting that GARP might be released in association with its own ligand. The high amount of TGF-β bound to sGARP was furthermore demonstrated in the reducing and denaturating SDS-PAGE (FIG. 6E).

Soluble GARP enhances TGFβ through αV integrins: It was next studied how cleaved GARP/latent TGF-β binds to the cell surface and enhances TGF-β activation. NMuMG SMAD2-GFP reporter cell line was stimulated with increasing sGARP concentrations. Remarkably, p-SMAD3 signaling increased in response to sGARP in a dose-dependent fashion, as indicated by GFP signal intensity (FIG. 7A). It was then asked how latent TGF-β in complex with GARP was able to elicit TGF-β signal transduction. Integrins has been extensively studied for their ability to bind and to activate TGF-β (Hinz 2013; Annes et al., 2004). In particular, αVβ6 and αVβ8 integrins have been shown to mediate TGF-β activation from membrane bound GARP in HEK293 and Treg cells, respectively. In 293 HEK, indeed, integrin αVβ6 binds to LAP's RGD sequence of the membrane bound GARP/latent TGF-β and helps to release mature TGF-β (Wang et al., 2012). It is thus plausible that integrins could mediate the binding and activation of latent TGF-β bound to sGARP. By flow cytometry and RT-PCR analysis it was observed that αV integrins are abundantly expressed on NMuMG cells (FIGS. 7B and C). The expression of the β chains of integrins that have been reported to activate TGF-β in vitro including αVβ6 (Munger et al., 1999), αVβ8 (Mu et al., 2002), αVβ5 (Wipff et al., 2007), and αVβ3 (Asano et al., 2005) was determined. Among the β integrins, β6 was the most expressed when compared to (33, (38, and 135 gene expression (FIG. 7C). Strikingly, increasing concentration of the RGD peptide was sufficient to decrease sGARP-dependent p-SMAD3 signaling in NMuMG, indicating that latent TGF-β mediates the binding between sGARP and integrins (FIG. 7D).

Soluble GARP/latent TGF-β complex is internalized by cells through integrins: Integrins binds to latent TGF-β on the cell surface where they predispose the complex for the release of the active peptide. Therefore, it was reasoned to detect soluble GARP complex on NMuMG cell surface. However, flow cytometry analysis and confocal pictures of NMuMG cells treated with soluble GARP or control IgG showed that after 1 hour of incubation soluble GARP is not present as bound to the cell membrane (FIG. 8A upper panel). Strikingly, it was observed that sGARP is internalized as represented by flow cytometry analysis and confocal pictures of permeabilized NMuMG cells (FIG. 8A lower panel). Integrins internalize ligands via receptor-mediated endocytosis. This is an interesting process that could give an alternative explanation to the integrin mediated surface release of active TGF-β (Weinreb et al., 2004). Integrins contribute to both binding and internalization of soluble GARP was investigated by confocal analysis that confirms first that soluble GARP is internalized, and second that this process is abrogated by RGD peptide, suggesting that LAP and integrins interaction mediates the endocytosis (FIG. 8B).

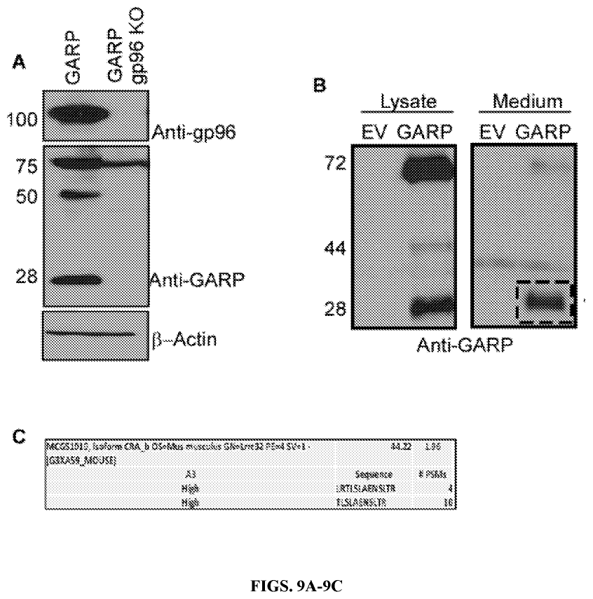

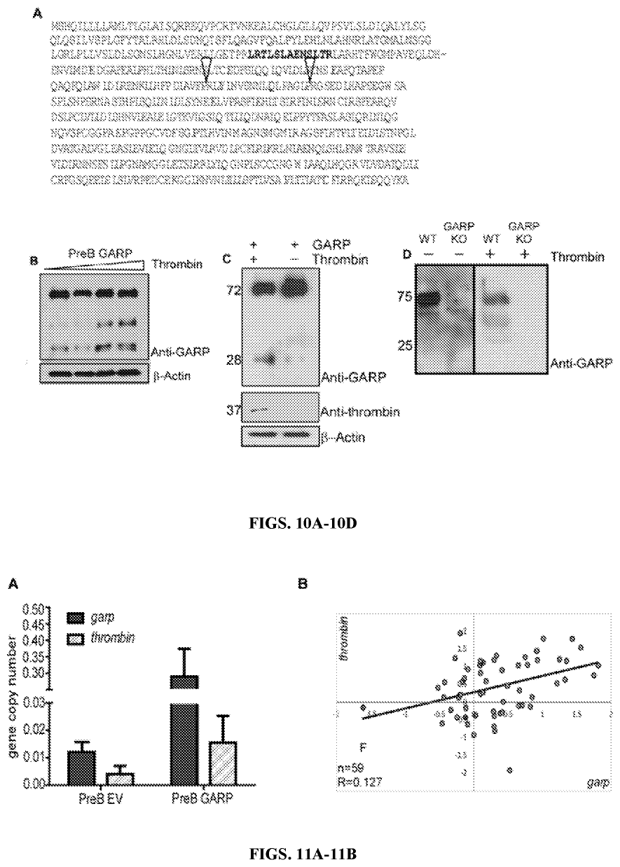

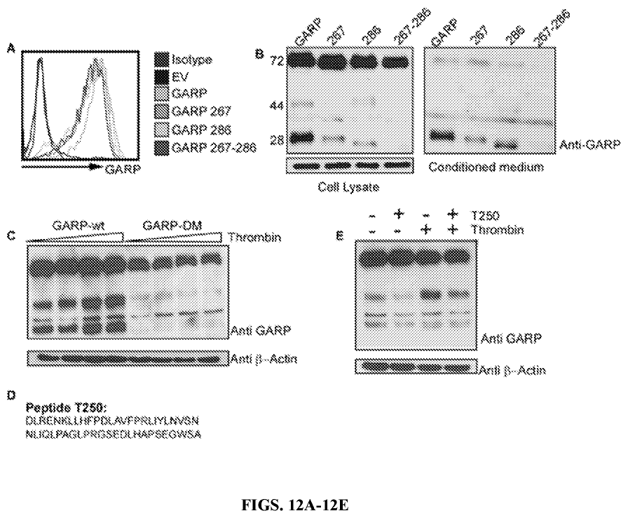

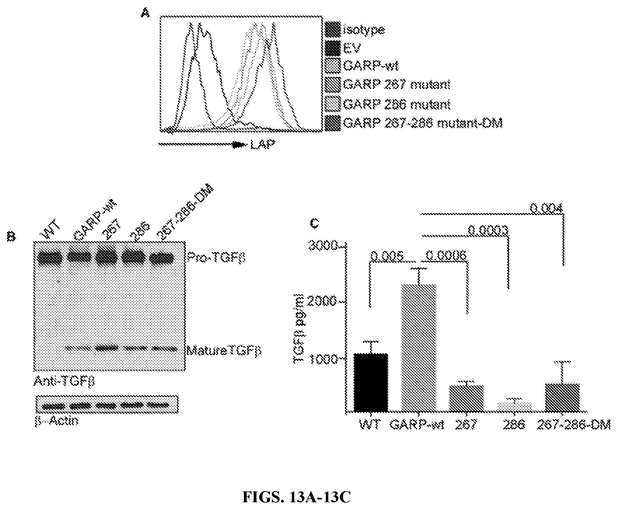

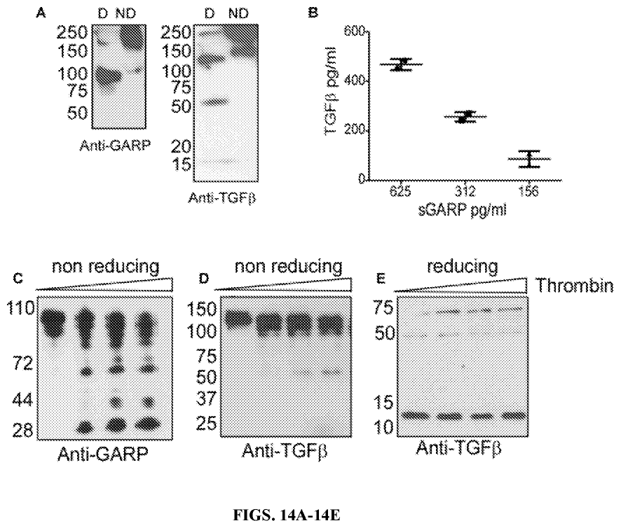

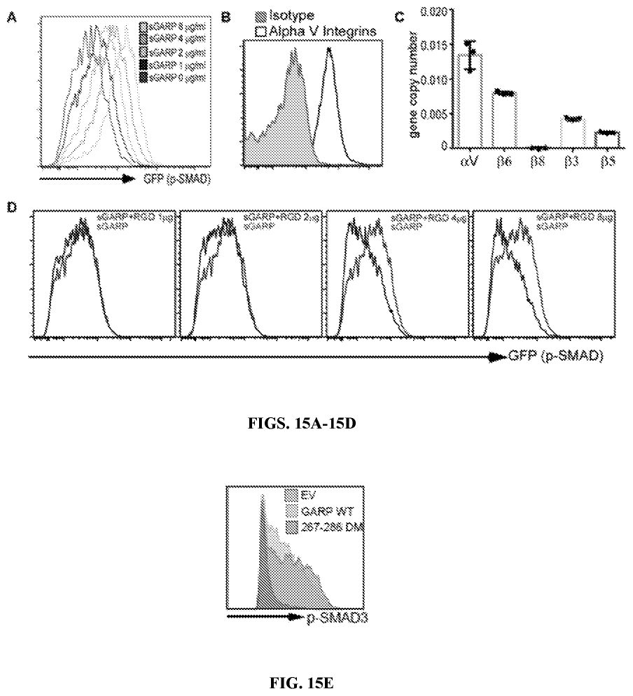

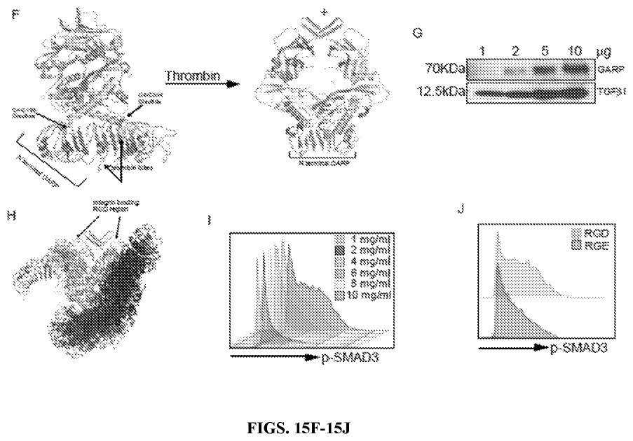

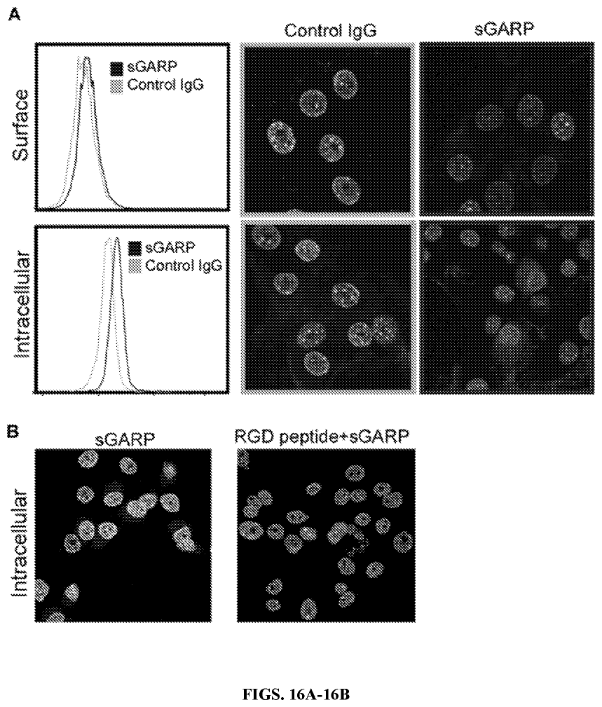

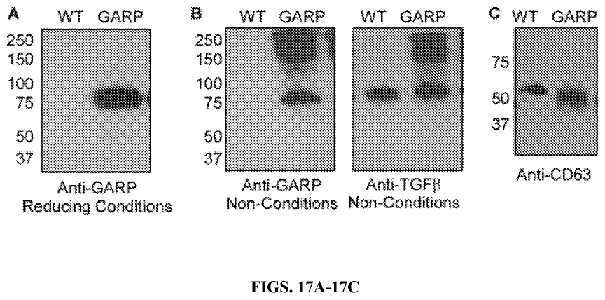

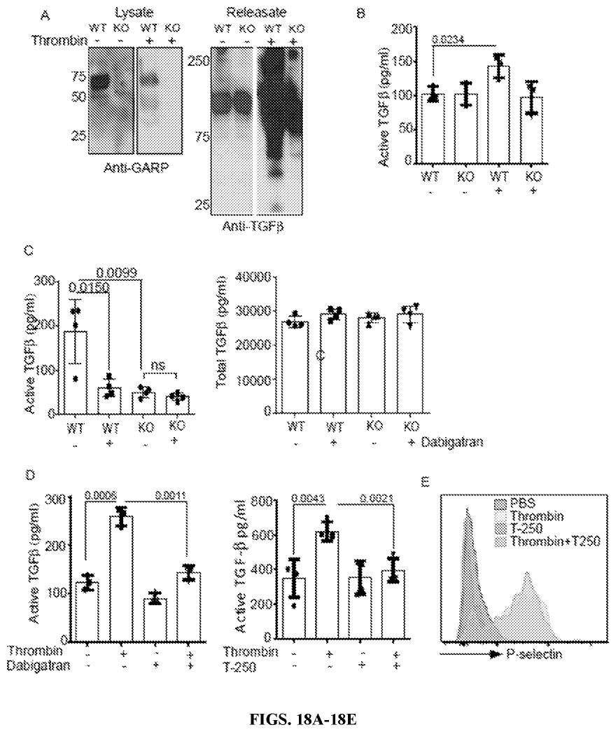

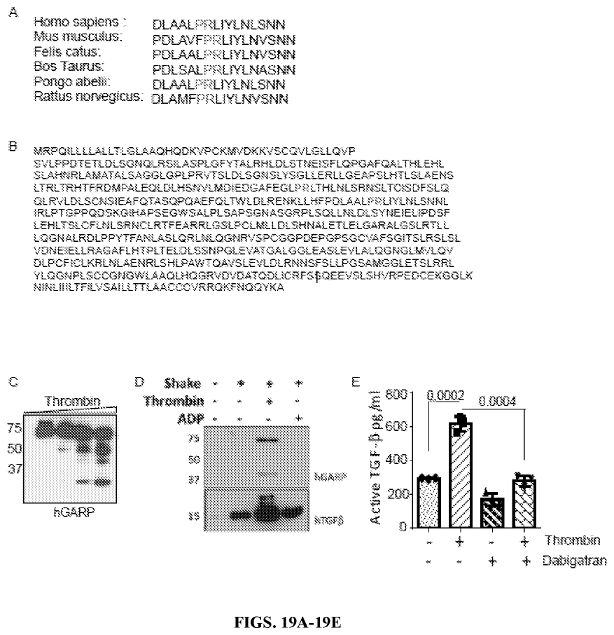

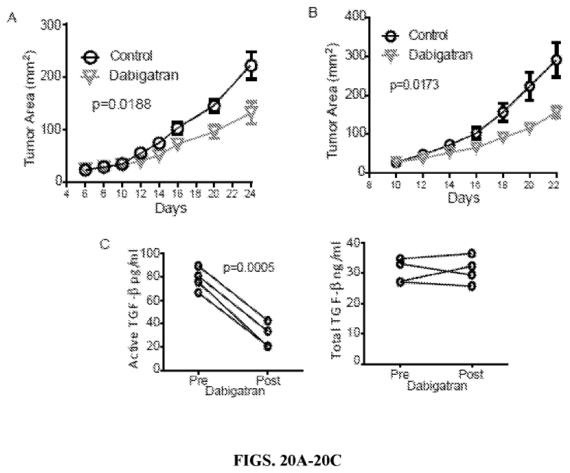

GARP/latent TGF-8 complex is released in exosomes: Western blot analysis of conditioned medium from GARP expressing cells indicated the presence of two cleaved products that, as shown earlier, are the results of thrombin mediated cleavage. Interestingly, a faint signal reflecting a full length of GARP was always detected, indicating that either the conditioned medium was contaminated by residues of whole cells, or GARP was released as a full length protein. The consistent detection of these full length protein prompted us to investigate if exosomes release constitutes an alternative mechanism of GARP secretion. It was hypothesized that GARP is an oncogenic protein that might be liberated via exosomes to then increase the metastatic potentials of the cancer cells. In support of this hypothesis, plasma collected from metastatic prostate patients revealed higher GARP concentration than non-metastatic patients. Additionally, following androgen deprivation therapy (ADT), there was an increase of soluble GARP concentration in plasma of prostate cancer patients in parallel with PSA1 decrease, suggesting GARP-rich exosomes release upon cancer cell death. Exosomes from 293 cells expressing TGF-β1 alone or in combination with GARP were isolated from serum free conditioned medium and analyzed by western blot. Strikingly, a consistent concentration of GARP was detected (FIG. 9A) and more importantly immunoblot for GARP and TGF-β in non-reducing and non-denaturating conditions reveals that exosomes contained GARP bound to TGF-β (FIG. 9B). The successful exosome isolations was proved by detection of the known exosome marker CD63 (FIG. 9C).

Herein, the studies showed evidence of two mechanisms that explain soluble GARP formation. The first involves the serine protease thrombin as the enzyme responsible to cleave surface GARP to generate two soluble products: a C-terminal bigger fragment and an N-terminal smaller one. It was found that this latter one is abundantly released in the extracellular milieu. Then, a recombinant GARP protein (sGARP) was generated lacking the transmembrane domain to mimic the N-terminal fragment that is released. It was observed that sGARP drives p-Smad3 phosphorylation by binding to surface integrins, through LAP. Additionally, the data show that integrins are responsible for sGARP cell internalization. These observations shed light on two possible mechanisms for active TGF-β release: integrins mediated surface TGF-β release, and endocytosis-mediated internalization of sGARP bound to integrins and subsequent signaling. Integrins indeed can be endocytosed to release their cargo in the endosomes where they can be either recycled or degraded (Bridgewater et al., 2012). The endocytosis-mediated mechanism of TGF-β activation might explain the absence of active TGF-β release upon thrombin cleavage (See FIG. 6D), yet the presence of Smad phosphorylation signaling elicited by soluble GARP on NMuMG cells. GARP dependent TGF-β activation indeed, has been mostly demonstrated by TGF-β reporter cell lines (mink lung epithelial reporter cells) and Smad 2/3 phosphorylation (Wang et al., 2012; Dedobbeleer et al., 2017).

### * * *

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Patent App. 60/133,116
U.S. Patent App. 60/135,818
U.S. Patent Pub. 2005/02506890
U.S. Patent Pub. 20140294898
U.S. Patent Pub. 2014022021
U.S. Patent Pub. 20110008369
U.S. Patent Pub. 2005/0260186
U.S. Patent Pub. 2006/0104968
U.S. Patent Pub. 2006/0008848
U.S. Pat. No. 4,682,195
U.S. Pat. No. 4,683,202
U.S. Pat. No. 4,797,368
U.S. Pat. No. 4,870,287
U.S. Pat. No. 5,139,941
U.S. Pat. No. 5,302,523
U.S. Pat. No. 5,322,783
U.S. Pat. No. 5,384,253
U.S. Pat. No. 5,440,013
U.S. Pat. No. 5,446,128
U.S. Pat. No. 5,464,765
U.S. Pat. No. 5,475,085
U.S. Pat. No. 5,538,877
U.S. Pat. No. 5,538,880
U.S. Pat. No. 5,550,318
U.S. Pat. No. 5,563,055
U.S. Pat. No. 5,580,859
U.S. Pat. No. 5,589,466
U.S. Pat. No. 5,591,616
U.S. Pat. No. 5,610,042
U.S. Pat. No. 5,618,914
U.S. Pat. No. 5,645,897
U.S. Pat. No. 5,656,610
U.S. Pat. No. 5,670,155
U.S. Pat. No. 5,672,681
U.S. Pat. No. 5,674,976
U.S. Pat. No. 5,702,932
U.S. Pat. No. 5,705,629
U.S. Pat. No. 5,710,245
U.S. Pat. No. 5,736,524
U.S. Pat. No. 5,739,169
U.S. Pat. No. 5,760,395
U.S. Pat. No. 5,780,448
U.S. Pat. No. 5,789,215
U.S. Pat. No. 5,801,005
U.S. Pat. No. 5,824,311
U.S. Pat. No. 5,830,880
U.S. Pat. No. 5,840,833
U.S. Pat. No. 5,844,905
U.S. Pat. No. 5,846,945
U.S. Pat. No. 5,859,184
U.S. Pat. No. 5,885,796
U.S. Pat. No. 5,889,155
U.S. Pat. No. 5,925,565
U.S. Pat. No. 5,928,906
U.S. Pat. No. 5,929,237
U.S. Pat. No. 5,935,819
U.S. Pat. No. 5,945,100
U.S. Pat. No. 5,981,274
U.S. Pat. No. 5,994,136
U.S. Pat. No. 5,994,624
U.S. Pat. No. 6,013,516
U.S. Pat. No. 6,207,156
U.S. Pat. No. 6,261,569
U.S. Pat. No. 6,740,320
U.S. Pat. No. 7,183,059
U.S. Pat. No. 7,192,713
U.S. Pat. No. 8,008,449
U.S. Pat. No. 8,017,114
U.S. Pat. No. 8,119,129
U.S. Pat. No. 8,329,867
U.S. Pat. No. 8,354,509
U.S. Pat. No. 8,735,553
PCT/US2017/025037
PCT/US00/14667
PCT/US99/11913
EP 266,032
WO94/09699
WO95/06128
WO1995001994
WO98/07408
WO98/42752
WO1998042752
WO2000037504
WO00/37504
WO01/14424
WO2001014424
WO2008/121767
WO2009/101611
WO2010/027827
WO2011/066342
WO2015016718
Aksentijevich et al., 1996
Alexander et al., 1988
Annes et al., The Journal of cell biology 165, 723-734, 2004.
Asano et al., Journal of immunology 175, 7708-7718, 2005.
Austin-Ward and Villaseca, 1998
Ausubel et al., 1996
Ausubel et al., eds., 1987
Baichwal and Sugden, 1986
Blomer et al., 1997
Bodanszky et al., 1976
Bridgewater et al., Journal of cell science 125, 3695-3701, 2012.
Bukowski et al., 1998
Caley et al., 1997
Camacho et al., J Clin Oncology 22(145): Abstract No. 2505 (antibody CP-675206), 2004.
Carter et al., 2008
Chen and Okayama, 1987
Christodoulides et al., 1998
Clark et al., 1995
Coffin, 1990
Cohen et al., 1990
Couch et al., 1963
Coupar et al., 1988
Davidson et al., 1998
Davis et al., 1996
Dedobbeleer et al., Journal of immunology, 2017.
di Bari et al., Cancer immunology, immunotherapy: CII 58, 1809-1818, 2009.
Edwards et al., Journal of immunology 190, 5506-5515, 2013.
Ercolani et al., 1988
Fechheimer et al., 1987
Felgner et al., 1987
Fischer, 2007
Flotte et al., 1995
Fraley et al., 1979
Friedmann, 1989
Froehler et al., 1986
Gabizon et al., 1990
Gauthy. et al. PloS one 8, e76186, 2013.
Ghosh and Bachhawat, 1991
Gopal, 1985
Graham and Prevec, 1991
Graham and Van Der Eb, 1973
Graham et al., 1977
Grunhaus and Horwitz, 1992
Hanibuchi et al., 1998
Harland and Weintraub, 1985
Haselmayer et al., Blood 110, 1029-1035, 2007.
Heller et al., 1996
Hellstrand et al., 1998
Hersdorffer et al., 1990
Hinz, B. Nature medicine 19, 1567-1568, 2013.
Hoffman, 1999
Hollander, 2012
Horwich et al., 1990
Hui and Hashimoto, 1998
Hurwitz et al., Proc Natl Acad Sci USA 95(17): 10067-10071, 1998.
Jackson, 1997
Johnson et al., 1993
Jones and Shenk, 1978
Jones et al., 1996
Jurasz et al., British journal of pharmacology 143, 819-826, 2004.
Jurk and Kehrel, The Journal of clinical investigation 125, 859-869, 2015.
Jurk and Kehrel, Semin Thromb Hemost 31, 381-392, 2005.
Kaeppler et al., 1990
Kaneda et al., 1989
Karin et al., 1989
Karlsson et al., 1986
Kato et al., 1991
Kirshmaier and Sugden, 1998
Kopp et al., Cancer research 69, 7775-7783, 2009.
Kotin et al., 1990
Laughlin, et al., 1986
Leal et al., 2014
Lebkowski, et al., 1988
Li, International journal of cancer. Journal international du cancer 138, 2078-2087, 2016.
Lindner, et. al., 2008
Liu et al., 1995
Macejak and Sarnow, 1991
Mann et al., 1983
Markowitz et al., 1988
McCarty et al., 1991
McLaughlin et al., 1988
Mellman et al., Nature 480:480-489, 2011.
Merrifield, 1963
Minskaia and Ryan, 2013
Mokyr et al., Cancer Res 58:5301-5304, 1998.
Mu et al., The Journal of cell biology 157, 493-507, 2002.
Munger et al., Cell 96, 319-328, 1999.
Muzyczka, 1992
Nabel et al, 1989
Naldini et al., 1996
Navia et al., 1992
Ng, 1989
Nicolas and Rubenstein, 1988
Nicolau and Sene, 1982
Nicolau et al., 1987
Pardoll, Nat Rev Cancer, 12(4): 252-64, 2012.
Paskind et al., 1975
Pelletier and Sonenberg, 1988
Peptide Synthesis, 1985
Philip et al., 1993
Philippe et al., The American journal of pathology 143, 1713-1723, 1993.
Potrykus et al., 1985
Potter et al., 1984
Protective Groups in Organic Chemistry, 1973
Qin et al., 1998
Quitsche et al., 1989
Racher et al., 1995
Rachidi et al., Science Immunolgy 2, 2017.
Remington's Pharmaceutical Sciences 22nd edition, 2012
Renan, 1990
Richards et al., 1984
Ridgeway, 1988
Rippe et al., 1990
Roux et al., 1989
Sambrook et al. 1989
Sambrook et al., 2001
Samulski et al., 1989
Samulski et al., 1991
Schafmeister et al., Journal of the American Chemical Society, 122(24): p. 5891-5892 (2000).
Shelling and Smith 1994.
Solid Phase Peptide Synthelia, 1984
Solodin et al., 1995
Stein et al., The American journal of medicine 119, 60-68, 2006.
Teicher 2009
Teicher 2014
Temin, 1986
Templeton et al. 1997
Thierry et al., 1995
Tiedt et al., Blood 109, 1503-1506, 2007.
Top et al., 1971
Tratschin, et al., 1984
Tsukamoto et al., 1995
Tur-Kaspa et al., 1986
Walensky et al., Science 305:1466-1470 (2004)
Wallace et al., Clinical cancer research: an official journal of the American Association for Cancer Research 14, 3966-3974, 2008.
Wang et al., Molecular biology of the cell 23, 1129-1139, 2012.
Weinreb et al., The Journal of biological chemistry 279, 2004.
Wilson et al., 1989
Wipff et al., The Journal of cell biology 179, 1311-1323, 2007.
Wong et al., 1980
Wu and Wu, 1987
Wu and Wu, 1988
Yang and Huang, 1997
Yang et al., 1994
Zhu el al., 1993
Zufferey et al., 1997

1-16. (canceled)

17. A method for treating cancer comprising administering an effective amount of an isolated peptide of comprising an amino acid sequence with at least 90% sequence identity to SEQ ID NO:1 or SEQ ID NO:54, wherein said peptide has a length of less than 100 residues and the sequence comprises two PR cleavage sites to a subject.

18. (canceled)

19. The method of claim 17, wherein the cancer is a breast cancer, lung cancer, head & neck cancer, prostate cancer, esophageal cancer, tracheal cancer, skin cancer brain cancer, liver cancer, bladder cancer, stomach cancer, pancreatic cancer, ovarian cancer, uterine cancer, cervical cancer, testicular cancer, colon cancer, rectal cancer, skin cancer (such as melanoma) or a hematological cancer.

20. (canceled)

21. The method of claim 17, wherein the cancer is metastatic.

22. The method of claim 17, wherein the peptide is administered systemically.

23. The method of claim 17, wherein the peptide is administered intravenously, intradermally, intratumorally, intramuscularly, intraperitoneally, subcutaneously, or locally.

24. The method of claim 1, wherein the cancer is a GARP positive cancer.

25. The method of claim 1, further comprising administering at least a second anticancer therapy to the subject.

26. The method of claim 25, wherein the second anticancer therapy is a surgical therapy, chemotherapy, radiation therapy, cryotherapy, hormonal therapy, immunotherapy or cytokine therapy.

27. The method of claim 25, wherein the second anticancer therapy comprises an immunotherapy, such as an immune checkpoint inhibitor, e.g., an anti-PD1 antibody.

28-29. (canceled)

30. The method of claim 27, wherein the immunotherapy comprises a T cell therapy.

31. The method of claim 27, wherein the immunotherapy comprises a T cell therapy and an immune checkpoint inhibitor.

32. (canceled)

33. A method for treating cancer in a subject comprising administering an effective amount of a direct thrombin inhibitor to the subject.

34-49. (canceled)

50. A method of treating cancer in a subject comprising administering an effective amount of an isolated peptide according to comprising an amino acid sequence with at least 90% sequence identity to SEQ ID NO:1 or SEQ ID NO:54, wherein said peptide has a length of less than 100 residues and the sequence comprises two PR cleavage sites and/or a direct thrombin inhibitor to the subject, wherein the subject is identified to have a high level of soluble GARP.

51-64. (canceled)
